(19) United States
(12) Patent Application Publication
(10) Pub. No.: US 2024/0401077 A1
(43) Pub. Date: Dec. 5, 2024

(54) KOALA RETROVIRUS ENVELOPE GYLCOPROTEINS AND USES THEREOF

(71) Applicant: Fraunhofer-Gesellschaft zur Förderung der angewandten Forschung e.V., Munchen (DE)

(72) Inventors: Stephan Fricke, Leipzig (DE); Dominik Schmiedel, Leipzig (DE); Alexander Renner, Leipzig (DE); Ulrike Köhl, Leipzig (DE)

(21) Appl. No.: 18/657,891

(22) Filed: May 8, 2024

Related U.S. Application Data
(63) Continuation of application No. PCT/EP2022/081009, filed on Nov. 7, 2022.

(30) Foreign Application Priority Data
Nov. 9, 2021 (EP) 21207080.9

(51) Int. Cl. C12N 15/86; C07K 14/005

(52) U.S. Cl. CPC C12N 15/86; C07K 14/005; C12N 2740/13022; C12N 2740/13052; C12N 2740/15043; C12N 2740/15052

(57) ABSTRACT

The present invention relates to an expression vector, an expression cassette or a pseudotyped viral vector particle comprising at least one nucleic acid encoding at least one Koala Retrovirus (KoRV) Envelope glycoprotein (Env), related nucleic acids, pseudotyped viral vector particles, mammalian packaging cell lines, transduced mammalian cells as well as related methods and uses.

### CROSS REFERENCE TO RELATED APPLICATIONS

This application is a Continuation of International Patent Application No. PCT/EP2022/081009, filed Nov. 7, 2022, which claims priority to European Patent Application No. 21207080.9, filed on Nov. 9, 2021. The contents of these applications are incorporated herein by reference in their entireties.

### SUBMISSION OF SEQUENCE LISTING XML

The content of the following submission on Sequence Listing XML is incorporated herein by reference in its entirety: a computer readable form (CRF) of the Sequence Listing (file name: F73842PC_sequence listing.XML, date created Sep. 7, 2022, size: 20,480 bytes).

### FIELD OF INVENTION

The present invention relates to an expression vector, an expression cassette or a pseudotyped viral vector particle comprising at least one nucleic acid encoding at least one Koala Retrovirus (KoRV) Envelope glycoprotein (Env), related nucleic acids, pseudotyped viral vector particles, mammalian packaging cell lines, transduced mammalian cells as well as related methods and uses.

### BACKGROUND

Gene therapy is defined as introducing a nucleic acid into target cells of a subject for therapeutic purposes, such as treating a disease. Various methods for the transient transfection are known, which may be used to genetically manipulate cells.

However, there are many obstacles, such as different degrees of efficiency, toxicity and off-target effects. One method that may be used is electroporation. Moreover, for a permanent and stable gene transfer, non-cell particles, such as viral vectors may be used. Viral vectors and related non-cell particles are generally capable of stably genetically manipulating cells in vitro, ex vivo, or in vivo. For example, gene therapy may be conducted in vivo or ex vivo or in vitro. In vitro (or ex vivo) gene therapy comprises the isolation of target cells from a subject, which are subsequently cultured, transduced with the desired nucleic acid carried by the viral vector and reintroduced into the subject, which may be the same or a different subject. This is also referred to as adoptive cell transfer. During in vivo gene therapy, the viral vector comprising the desired nucleic acid may be directly injected into the subject to be treated and transduction takes place in the subject's body. Moreover, stable gene transfer to mammalian cells is also desirable for many in vitro purposes, such for research, diagnostic or monitoring purposes.

However, also viral vectors still bear several challenges, such as insertional mutagenesis caused by gammaretroviral vectors, that may occur during optimization of viral vector-based gene transfer. Therefore, for example, lentiviral vectors are a commonly used tool to genetically modify cells. They usually comprise a lentivirus capsule covered by envelope glycoproteins that may interact with the surface of the target cells. Inside, they carry the viral vector comprising with the nucleic acid of interest.

Pseudotyping plays a major role in improvement of transduction levels of mammalian cells, e.g. hematopoietic target cells. Pseudotyping means that a virus or virus particle has a modification to one or more of its envelope proteins, such as its glycoproteins. For example, the glycoproteins are replaced by glycoproteins of other enveloped viruses. This allows the targeting of other target cells than those that are usually the destination of glycoproteins of the respective virus. Viral envelope glycoproteins (Env) that are generally widely used for pseudotyping of lentiviral vectors are e.g. Env derived from or based on vesicular stomatitis virus (VSV), feline leukemia virus (FeLV, also known as RD114), measles virus, Nipah virus, cocal virus or Baboon endogenous retrovirus (BaEV).

Lentiviral vectors pseudotyped with BaEV Env were previously described in Girard-Gagnepain et al. 2014, Bari et al., 2019, Colamartino et al., 2019; WO2013/045639 A1 and WO2019/121945 A1.

However, there are still specific cell types that are difficult to modify, even with viral vectors, such as above-mentioned lentiviral vectors pseudotyped with BaEV Env. This makes the development of gene therapies targeting such cells very challenging. Examples of such cells are mammalian cells, such as hematopoietic cells, iPS (induced pluripotent stem) cells, stem cells, such as hematopoietic cells, or immune cells (e.g. T cells, NK cells, B cells, dendritic cells, monocytes or macrophages).

However, modification of above-mentioned cell types would have many benefits. For example, hematopoietic stem cells are desired target cells in gene therapy as their genetic modification would be transferred to any lineages derived from them. Targeting T cells or NK cells would allow the development of Chimeric antigen receptor T cells (CAR-T cells) and Chimeric antigen receptor natural killer cells (CAR-NK cells) having the ability to target a specific protein, e.g. on cancer cells. Natural killer (NK) cells are part of the innate system and may kill virus-infected and tumor cells without need of stimulation.

For example, even for lentiviral vectors pseudotyped with BaEV, activation of NK cells is required prior to transduction.

For the development of innovative immune therapies, such as cell and gene therapies, efficient tools for the genetic manipulation of immune cells are necessary. Therefore, there is a need to improve the transduction process itself in order to increase the percentage of gene modified cells and thus efficacy of the gene therapy. A suitable transduction tool should be able to deliver its cargo efficient, fast and precise, it should be non-toxic to the cells and should require minimum manipulation of the cells. If possible, excessive in vitro culturing and pre-activation prior to transduction should be avoided.

The expression vector, expression cassette or pseudotyped viral vector particle of the present invention comprising at least one nucleic acid encoding at least one Koala Retrovirus (KoRV) Envelope glycoprotein (Env) solve all above-mentioned problems and are surprisingly advantageous in genetically manipulating mammalian cells of interest.

### SUMMARY OF THE INVENTION

The above-mentioned objects have been solved by the aspects of the present invention as specified hereinafter.

In detail, it has been found that Koala Retrovirus (KoRV) Envelope glycoproteins (Env) KoRVA and KoRVB as well as their combination KoRVAB allow the production of viral vector particles containing a transfer gene of interest. Further, it has been found that lentiviral vector particles enveloped with KoRVA or KoRVB protein as well as their combination (designated herein as “KoRVAB”) are suitable for transduction. Especially and very surprisingly, it was found that the transgene transfer for NK cells is extremely efficient with KoRVA-, KoRVB- or KoRVAB-pseudotyped lentiviral vector particles and was even better than previously described viral envelopes, including BaEV in particular as no pre-activation of NK cells with a cytokine is required (see FIGS. 3 and 4).

An expression vector, an expression cassette or a pseudotyped viral vector particle comprising at least one nucleic acid encoding at least one Koala Retrovirus (KoRV) Envelope glycoprotein (Env) is provided.

Moreover, a nucleic acid encoding at least one KORV Env which lacks the fusion inhibitory R-peptide (R-peptide) or lacks part of the R-peptide is provided.

Also provided is a pseudotyped viral vector particle which is pseudotyped with at least one KoRV Env as well as a mammalian packaging cell line producing the pseudotyped viral vector particle is provided.

Further, an in vitro method for delivery of at least one payload nucleic acid to at least one mammalian cell is provided as well as a transduced mammalian cell obtainable by such method.

Finally, an in vitro use of at least one KoRV Env glycoprotein, or of at least one nucleic acid encoding a KoRV Env glycoprotein, or of a mammalian packaging cell line, an expression vector or expression cassette, a pseudotyped viral vector particle or a nucleic acid is provided.

### DESCRIPTIONS OF THE FIGURES

FIG. 1: Schematic overview of the protein domains of full-length envelope glycoprotein KoRVA Env (top) and modified envelope glycoprotein KORVA Env (bottom). In detail, both forms comprise an N-terminal signal peptide at amino acid positions 1-35, an extracellular domain at amino acid positions 36-606, a transmembrane domain at amino acid positions 607-627 as well as a cytoplasmic domain at their C-terminus. While the full-length envelope glycoprotein KoRVA Env (top) comprises the complete cytoplasmic domain at amino acid positions 628-659, which comprises a C-terminal R-peptide at amino acid positions 645-659, the modified envelope glycoprotein KoRVA env (bottom) lacks such C-terminal R-peptide and therefore only comprises amino acid positions 628-644 of the cytoplasmic domain. In the pseudotyped viral vector particles generated therefrom, the signal peptide is removed. The pseudotyped viral vector particles comprise mature a KoRV Env.

FIG. 2: Schematic overview of pseudovirus production in HEK293T cells as well as subsequent NK cell transduction and transgene expression in NK cells. In detail, the pseudovirus may be generated by transfection with suitable viral helper plasmid(s) (encoding genes such as gag and pol), a viral vector carrying the specific payload nucleic acid (here designated as transgene, such as, e.g., a CAR, green fluorescent protein (GFP), etc.), and at least one vector comprising a KoRV envelope protein. Viral vectors carrying such features are used to transfect HEK293T cells, which subsequently produce pseudoviruses comprising the KoRV envelope protein and a viral genome comprising the transfer vector (i.e. pseudotyped viral vector particles of the invention). The pseudotyped viral vector particles produced in this way are added to mammalian cells of interest, such as purified NK cells. This can be performed using a commercially available transduction enhancer (e.g. Vectofusin, Miltenyi Biotec GmbH, Bergisch Gladbach) to transfer the transfer gene into these mammalian cells (such as immune cells).

FIG. 3: Transduction efficiency in peripheral blood NK cells of 3 different donors. Isolated NK cells were transduced on the day of isolation, alternatively on day 3, 7 and 21 after activation in NK MACS medium (Miltenyi) containing 500 U/mL IL-2 and 140 U/mL IL-15 (Peprotech), with KoRV- or BaEV-pseudotyped lentiviral vector particles. Transduction efficiency was determined 72 hours after transduction by flow cytometric analysis of the fluorescent protein mVenus.

FIG. 4: Transduction efficiency in fresh, primary NK cells.

NK cells isolated from peripheral blood were transduced with KoRV- or BaEV-pseudotyped lentiviral vector particles on the day of isolation. Transduction efficiency was determined 72 hours after transduction by flow cytometric analysis of the fluorescent protein mVenus. The transduction efficiency averaged over three donors and the standard deviation are plotted. Statistical analysis was performed using one-way analysis of variance (ANOVA), *=p<0.05.

FIG. 5: Transduction of peripheral blood mononuclear cells (PBMCs) with KORV-A- or KoRV-B-enveloped lentiviruses.

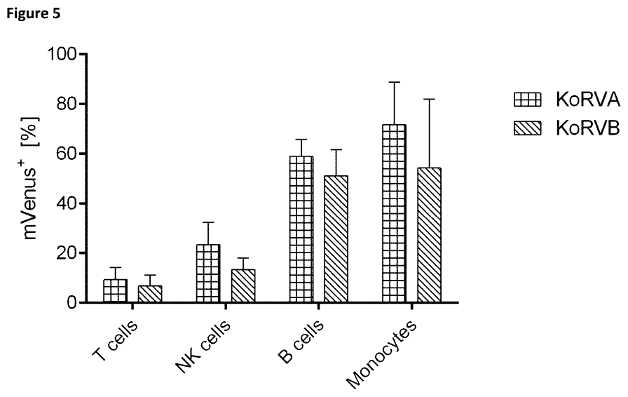

PBMCs from peripheral blood of 6 healthy donors were isolated and immediately transduced (without separating the immune cell populations). Using the fluorescent reporter gene mVenus, transduction efficiencies were determined in CD19+ B cells, CD14+ monocytes, CD56+/CD3-NK cells and CD3+ T cells 3 days after transduction.

### DETAILED DESCRIPTION OF THE INVENTION

The present invention provides an expression vector, an expression cassette or a pseudotyped viral vector particle comprising at least one nucleic acid encoding at least one Koala Retrovirus (KoRV) Envelope glycoprotein (Env).

Koala Retrovirus (KoRV) is an enveloped retrovirus belonging to the genus of gammaretroviruses. So far, the only known target organism of KoRV are koalas, where it may cause the koala immune deficiency syndrome (KIDS), an immunodeficiency disease. As other retroviruses, KoRV comprises a viral envelope, which constitutes the outer layer of the virus encompassing the nucleic acid material of that virus. Among other components, the viral envelope of KORV comprises Envelope glycoproteins (Env) of which so far several variants are known, such as KoRVA and KoRVB. For example, KoRV Env sequences described or postulated in the art include KoRVA Env, KoRVB Env, KoRVC Env, KoRVD Env, KoRVE Env, KoRVF Env, and KoRVJ Env. Such variants may e.g. target different host cell proteins. For example, KoRVA has been shown to use a phosphate transporter (PiT1) for cell attachment and entry, while KoRVB infects via SLC19A2.

The term “KoRV Env” further includes chimeras of such naturally occurring variants, such as, for example, a chimera of KoRVA Env and KoRVB Env. For example, the KORV Env chimera may be independently assembled from domains from at least two naturally occurring variants. The domains of Env include the signal peptide, the extracellular domain, the transmembrane domain, and the cytoplasmic domain.

The cytoplasmic domain preferably lacks the fusion inhibitory R-peptide (R-peptide) or lacks part of the R-peptide.

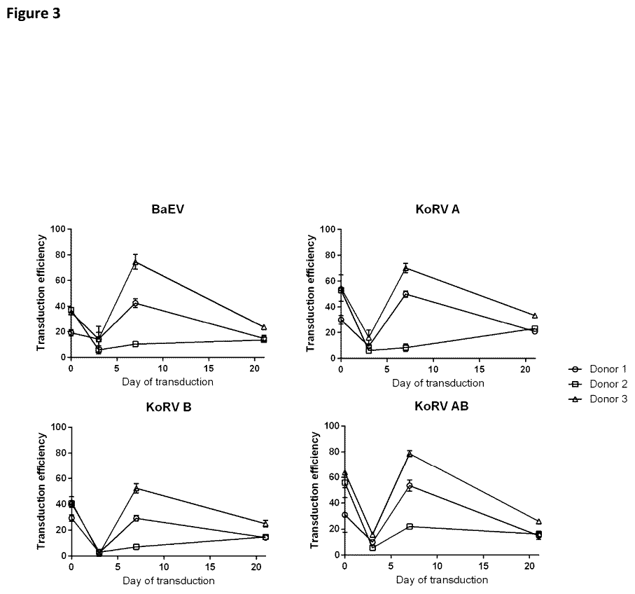

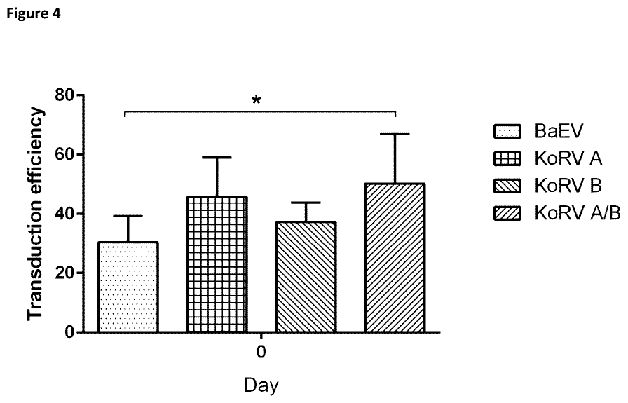

As shown in FIGS. 3 and 4, efficient transduction of NK cells, which are known to be difficult to transduce, can be achieved using either KoRVA lacking the R-peptide, KoRVB lacking the peptide, or a mixture of KoRVA lacking the R-peptide and KoRVB lacking the peptide for preparing pseudotyped viral vector particles.

In the context of the invention, the expressions “fusion inhibitory R peptide”, “C-terminal fusion inhibitory R peptide”, “R-peptide” and “C-terminal R-peptide”, are used as synonyms and refer to the C-terminal portion of the cytoplasmic tail domain of the envelope glycoprotein which is cleaved by a viral protease during virion maturation, thus enhancing membrane fusion of the envelope glycoprotein.

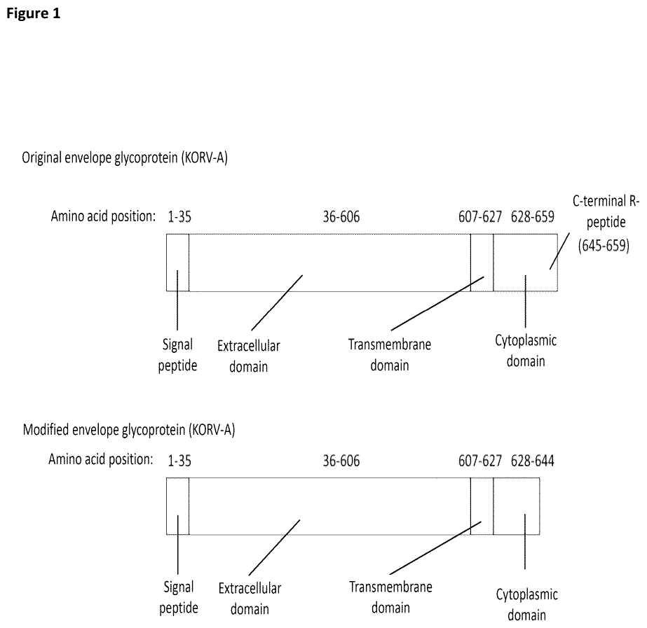

The fusion inhibitory R peptide of the KoRVA Env protein corresponds to amino acids 645 and 659 of the full-length wild-type KoRVA Env sequence, as shown in FIG. 1. The full-length wildtype KoRVA Env sequence is shown in SEQ ID No: 6.

The fusion inhibitory R peptide of the KoRVB Env protein corresponds to amino acids 652 to 666 of the full-length wildtype KoRVB Env sequence. The full-length wildtype KoRVB Env sequence is shown in SEQ ID No: 8.

The expression vector, expression cassette or pseudotyped viral vector particle comprises at least one nucleic acid encoding at least one such KoRV Env.

The term “nucleic acid”, as used herein, generally describes any form of deoxyribonucleic acid (DNA), ribonucleic acid (RNA) or artificial nucleic acid known to the person skilled in the art, such as e.g. peptide nucleic acid (PNA).

In one embodiment, the nucleic acid is DNA. In one embodiment, the nucleic acid is RNA.

Nucleic acid sequences coding for a Koala Retrovirus (KoRV) Envelope glycoprotein (Env) described herein, and modified versions of these proteins, can be determined using methods well known in the art, i.e., nucleotide codons known to encode particular amino acids are assembled in such a way to generate a nucleic acid that encodes the Env protein. Such a nucleic acid (or polynucleotide) encoding the Env protein can be assembled from chemically synthesized oligonucleotides, which, briefly, involves the synthesis of overlapping oligonucleotides containing portions of the sequence encoding the Env protein, annealing and ligating of those oligonucleotides, and then amplification of the ligated oligonucleotides by PCR. Alternatively, generation and/or amplification of the desired nucleic acid sequences can be achieved using amplification techniques such as PCR methods.

When a nucleic acid encoding a KoRV Env is not available, but the amino acid sequence of the protein molecule is known, a nucleic acid encoding the KoRV Env can be chemically synthesized or obtained from a suitable source e.g., a nucleic acid, isolated from any tissue or cells expressing the protein by PCR amplification using synthetic primers hybridizable to the 3′ and 5′ ends of the sequence or by cloning using an oligonucleotide probe specific for the particular gene sequence to identify, e.g., a DNA clone that encodes the Env protein. Amplified nucleic acids generated by PCR can then be cloned into replicable cloning vectors using any method well known in the art.

The nucleic acid as contained in the expression vector, expression cassette or pseudotyped viral vector particle comprises at least one nucleic acid encoding at least one such KoRV Env.

Preferably, the expression vector, expression cassette or pseudotyped viral vector particle comprises one nucleic acid encoding at least one such KoRV Env. Preferably, the expression vector, expression cassette or pseudotyped viral vector particle comprises one nucleic acid encoding one such KORV Env. Preferably, the KoRV is selected from KoRVA and KoRVB. In another preferred embodiment, the expression vector, expression cassette or pseudotyped viral vector particle comprises one nucleic acid encoding two or more KoRV Env, such as 2, 3, 4, 5, 6 7, or more KoRV Env. Preferably, the two or more KoRV Env comprise or consist of a KoRVA and a KoRVB Env.

Preferably, the nucleic acid as contained in the expression vector, expression cassette or pseudotyped viral vector particle comprises at least two such KoRV Env, at least three such KORV Env or at least four such KoRV Env.

These KoRV Env may for example differ in that they originate from different envelope glycoprotein variants, such as KoRVA and KoRVB, they may be chimeras of such variants or may differ in being mutational variants of the same envelope glycoprotein variant.

The KoRV Env protein of the invention therefore also encompasses a KoRV Env protein, which contains 1, 2, 3, 4, 5, 6, 7, 8, 9, 10, 11, 12, 13, 14, 15, 16, 17, 18, 19 or 20 mutations as compared to a full-length or mature wildtype KoRV protein, which optionally lacks the C-terminal R-peptide or part of the C-terminal R-peptide. The KORV Env protein of the invention therefore encompasses a KoRV Env protein, which contains 1, 2, 3, 4, 5, 6, 7, 8, 9, 10, 11, 12, 13, 14, 15, 16, 17, 18, 19 or 20 mutations as compared to a KoRV protein as shown in any of SEQ ID Nos: 2, 4, 6 and 8 or a mature form thereof.

The nucleic acids of the invention therefore also encompass nucleic acids encoding at least one KoRV Env protein, which contains 1, 2, 3, 4, 5, 6, 7, 8, 9, 10, 11, 12, 13, 14, 15, 16, 17, 18, 19 or 20 mutations as compared to a full-length or mature wildtype KoRV protein, which optionally lacks the C-terminal R-peptide or part of the C-terminal R-peptide. The nucleic acids of the invention therefore encompass nucleic acids encoding at least one KoRV Env protein, which contains 1, 2, 3, 4, 5, 6, 7, 8, 9, 10, 11, 12, 13, 14, 15, 16, 17, 18, 19 or 20 mutations as compared to a KoRV protein as shown in any of SEQ ID Nos: 2, 4, 6 and 8 or a mature form thereof.

It is also possible to use truncated and full-length KoRV Env proteins of the same variant, such as, e.g. a nucleic acid encoding full-length KoRVA Env and a nucleic acid encoding KoRVA Env lacking the R-peptide or part thereof, or, e.g. a nucleic acid encoding full-length KoRVB Env and a nucleic acid encoding KoRVB Env lacking the R-peptide or part thereof.

In the pseudotyped viral vector particles of the invention, the signal peptide is removed from KoRV Env proteins. The pseudotyped viral vector particles of the invention comprise mature KoRV Env proteins. Preferably, the mature KoRV Env protein lacks the R-peptide or lacks part of the R-peptide. Alternatively, the mature KoRV Env protein comprises the complete cytoplasmic domain.

The KoRV proteins of the invention or KoRV proteins encoded by the nucleic acids of the invention are preferably able of binding to and fusing with at least one mammalian cell membrane, thereby transferring biological material into said activated NK cells. Methods for determining the binding to and fusing with at least one mammalian cell membrane are well known in the art. For example, the ability to transduce a mammalian cell with a payload nucleic acid, such as a human NK cell, can be determined as shown in the examples.

In the first aspect of the invention, such nucleic acid encoding at least one KoRV Env is part of an expression vector, expression cassette or pseudotyped viral vector particle.

In general, such expression vector, expression cassette or pseudotyped viral vector particle comprises at least one nucleic acid encoding at least one KoRV Env. Moreover, the expression vector, expression cassette or pseudotyped viral vector particle may also comprise at least two nucleic acids encoding at least one KoRV Env, at least three nucleic acids encoding at least one KoRV or at least four nucleic acids encoding at least one KoRV. For example, the expression vector, expression cassette or pseudotyped viral vector particle may also comprise at least two nucleic acids encoding a KoRVA Env. For example, the at least two nucleic acids encoding a KoRVA Env may include e.g. a nucleic acid encoding a full-length KoRVA Env and a nucleic acid encoding KoRVA Env lacking the R-peptide or part thereof. For example, the expression vector, expression cassette or pseudotyped viral vector particle may also comprise at least two nucleic acids encoding a KoRVB Env. For example, the at least two nucleic acids encoding a KoRVB Env may include e.g. a nucleic acid encoding a full-length KoRVB Env and a nucleic acid encoding KoRVB Env lacking the R-peptide or part thereof.

Preferably, the expression vector, expression cassette or pseudotyped viral vector particle may also comprise at least two nucleic acids encoding a KoRV Env, wherein at least one KoRV Env encodes a KoRVA Env and at least one KoRV Env encodes a KoRVB Env. Preferably, both KORV Env lack the R-peptide or part of the R-peptide.

The term “expression vector”, as used herein, means plasmids which are used to introduce a desired nucleic acid sequence, such as a gene, into a target cell, resulting in the transcription and translation of the protein encoded by the nucleic acid sequence, e.g. KoRV Env. Therefore, an expression vector in general comprises regulatory sequences, such as promoter and enhancer regions, as well as, optionally, a polyadenylation site in order to direct efficient transcription of the nucleic acid sequence on the expression vector. The expression vector may further comprise additional necessary or useful regions, such as a selectable marker for selection in eukaryotic or prokaryotic cells, a purification tag for the purification of the resulting protein, a multiple cloning site or an origin of replication.

Usually, the expression vector may be a viral or a non-viral vector. In general, various kinds of viral vectors, such as retroviral vectors, e.g. lentiviral, gammaretroviral or adenoviral vectors, or plasmids may be used as expression vectors. For example, plasmids for expression in bacteria may be used for cloning and amplification or sequences and expression cassettes of the invention. For example, vectors can be suitable for replication and integration in mammalian cells. Further, cloning vectors may contain transcription and translation terminators, initiation sequences, and promoters useful for expression of the desired nucleic acid sequence. In case of a plasmid, the plasmid preferably comprises a promoter suitable for expression in a cell.

The term “expression cassette”, as used herein, means any DNA segment suitable for the synthesis of the corresponding RNA, which subsequently may be translated to the corresponding protein. Therefore, it usually comprises a protein-coding sequence, which in case of the present invention usually is a nucleic acid sequence encoding a KoRV Env protein as well as regulatory sequences allowing regulation of the transcription. In detail, such regulatory sequences may for example comprise a promoter region coding for the starting point of the transcription, an open reading frame (comprising the protein-coding sequence, or the RNA of interest), a binding site for ribosomes, a terminator region coding for the ending point of transcription or a 3′ untranslated region (such as a polyadenylation site).

For expression of a KoRV Env of the invention, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Expression of nucleic acids is typically achieved by operably linking a nucleic acid encoding the gene of interest to a promoter and incorporating the construct into an expression vector.

In general, the expression cassette according to the first aspect of the invention comprises at least a protein-coding sequence, which in case of the present invention is a nucleic acid sequence encoding a KoRV Env protein, as well as a promoter region. Optionally, the expression cassette according to the first aspect of the invention may additionally comprise an open reading frame, a binding site for ribosomes, a terminator region and/or a 3′ untranslated region (such as a polyadenylation site).

Suitable regulatory elements that may be used in the expression cassette according to the first aspect of the invention are well-known by the person skilled in the art and may, for example, may be chosen based on the type of organism of the host cell the expression cassette is intended to be transfected to. For example, such regulatory elements may originate from mammalian cells (e.g. from mammalian packaging cell lines), bacteria, yeast, plants and/or insects. Preferably, suitable regulatory elements that may be used in the expression cassette according to the first aspect of the invention originate from mammalian cells.

The term “viral vector particle”, as used herein, means any virus particle or viral-like particle. A “viral vector particle” comprises a KoRV Env protein on its surface. Thereby, a “virus particle”, as used herein, means any infectious agent capable of infecting at least one living cell, preferably at least one mammalian cell.

The term “viral-like particle”, as used herein, differs from a “virus particle” in that it is non-infectious due to lacking viral genetic material that would allow an infection. Examples for suitable virus particles or viral-like particles are well-known to the person skilled in the art.

For example, the viral vector particle or viral-like particle may be a retroviral vector particle or retroviral-like particle (e.g. a lentiviral vector particle or lentiviral-like particle or gammaretroviral vector particle or gammaretroviral-like particle).

Virus-like particles (VLPs) resemble viral particles, but are not infecting or transducing because they contain no viral genetic material encoding for the proteins of the virus-like particle. In particular, VLPs in the context of retroviral vectors do not contain psi positive nucleic acid molecules. The expression of viral structural proteins, such as the Env, can result in the assembly of virus like particles (VLPs). Like for viral vector particles, VLPs can also be pseudotyped using the KoRV Env of the invention. VLPs may be used to deliver target nucleic acids to the cytoplasm of target cells. In particular, VLPs are useful as vaccines.

For example, the expression vector may be a viral vector, preferably, a lentiviral vector (e.g., a lentiviral expression vector) or a gammaretroviral vector (e.g., a gammaretroviral expression vector). The viral vector may comprise a lentiviral transfer plasmid (e.g., as naked DNA) or may be comprised in an infectious virus particle, such as a lentiviral virus particle or a gammaretroviral virus particle.

With respect to elements such as cloning sites, promoters, regulatory elements, heterologous nucleic acids, etc., it is to be understood that the sequences of these elements can be present in RNA form in lentiviral or gammaretroviral vector particles and can be present in DNA form in DNA plasmids.

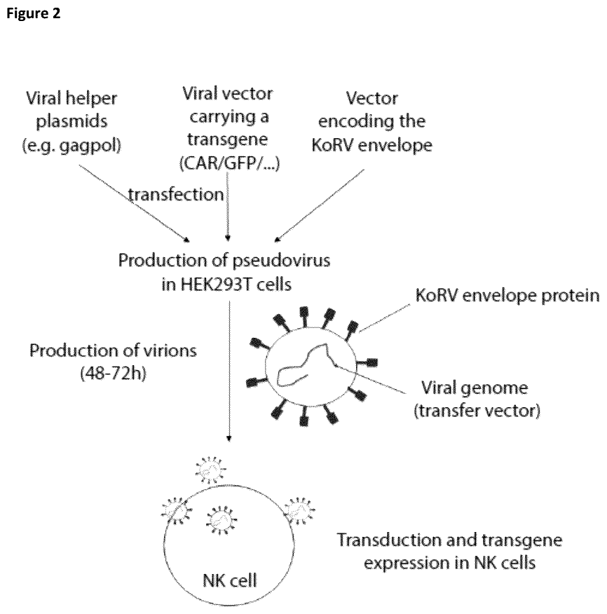

The term “pseudotyped viral vector particle”, as used herein, means any pseudotyped viral vector particle. The term “pseudotyped”, as used herein, means that a viral vector particle or viral-like particle has a modification to one or more of its envelope proteins, e.g., an envelope protein is substituted with an envelope protein from another virus. For example, in case of the present invention, this means that a viral vector particle or viral-like particle, which is not originating from Koala Retrovirus, comprises a KoRV Env protein, such as KoRVA or KoRVB, or a mixture thereof. This may, for example, be achieved as shown in FIG. 2. In detail, the nucleic acid encoding the naturally occurring envelope proteins of that virus may be replaced by nucleic acids encoding KoRV Env protein. Alternatively, nucleic acids encoding KoRV Env protein may also be introduced into such natural viral expression vector without deleting the nucleic acid sequences encoding for the natural envelope proteins of that virus. The resulting viral expression vector may then be transfected to cells, which subsequently produce pseudotyped viral vector particles comprising the KoRV envelope protein and a viral genome comprising the transfer vector.

It is understood that in 2^{nd} and 3^{rd} generation of viral expression systems, in particular, retroviral, more preferably, lentiviral and gammaretroviral vector systems, parts of the viral genome may be deleted and/or is present on different expression vectors. For example, the at least one KoRV nucleic acid may be present on one expression plasmid. For example, a further viral vector may comprise gag, pol, and/or RRE and optionally Rev. In an alternative embodiment, Rev may be present on a separate viral vector. Further, a further expression vector, designated transfer vector, may comprise the payload nucleic acid.

For example, 3^{rd} generation lentiviral or gammaretroviral packaging plasmids may include expression vectors expressing (i) gag and pol, (ii) rev, and (iii) at least one KoRV protein, and transgene transfer plasmid comprising at least one payload nucleic acid.

However, also different arrangements of the elements on expression plasmids is possible to produce pseudotyped viral vector particles.

For example, WO2021/041322 discloses method which utilize two plasmids, rather than four, to provide the required packaging elements and transfer vector to a cell, thereby producing a packaging cell line of the invention. In such method, applied to the present technology, a mammalian cell is transfected with the following: i. a packaging vector including an expression cassette, encoding: 1. a lentiviral regulator of expression of virion proteins (REV) gene under control of a first promoter; 2. a lentiviral envelope gene under control of a second promoter; and 3. a lentiviral group specific antigen (GAG) gene and a lentiviral polymerase (POL) gene both under control of a third promoter, wherein the expression cassette is flanked on both the 5′ and 3′ ends by transposon-specific inverted terminal repeats (ITR); and ii. a transfer vector, comprising: 1. a nucleic acid sequence encoding a gene of interest under control of a fourth promoter, wherein the nucleic acid sequence is flanked on both the 5′ and 3′ ends by transposon-specific inverted terminal repeats (ITR). Applying the method to the present invention, the lentiviral envelope gene is replaced by a KoRV Env nucleic acid of the invention.

An alternative method with a yet different arrangement of plasmids as expression vectors which can be used is described in WO2021/127076. The methods utilize three plasmids, rather than four, to provide the required packaging elements and transfer vector to a cell. In such alternative method, applied to the present invention, a mammalian cell is transfected with the following: i. a first nucleic acid encoding a lentiviral regulator of expression of virion proteins (REV) gene under control of a first promoter and an envelope glycoprotein gene under control of a second promoter; ii. a second nucleic acid encoding a gene of interest under control of a third promoter; and iii. a third nucleic acid encoding a lentiviral group specific antigen (GAG) gene and a lentiviral polymerase (POL) gene both under control of a fourth promoter. Applying the method to the present invention, the lentiviral envelope gene is replaced by a KoRV Env nucleic acid of the invention.

Accordingly, the term “pseudotyped viral vector particle” refers to a viral vector particle comprising a viral envelope glycoprotein which is exogenous to the vector.

“Exogenous” is understood as that the protein or agent is neither comprised by nor encoded in the corresponding wildtype virus from which the viral vector particle is derived, such as a lentivirus or gammaretrovirus.

The viral vectors according to the invention are pseudotyped with at least one KoRV Env.

Preferably, the pseudotyped viral vector particle is a lentiviral or gammaretroviral vector particle, and the viral vector particle with at least one KoRV Env. Such pseudotyped viral vector particle is for example shown in FIG. 1.

A viral vector comprises a nucleic acid molecule, such as a transfer plasmid, that includes virus-derived nucleic acid elements that typically facilitate transfer of the nucleic acid molecule or integration into the genome of a cell or to a viral vector particle that mediates nucleic acid transfer. Viral vector particles will typically include various viral components and sometimes also host cell components in addition to nucleic acid(s).

A gammaretroviral vector can comprise a viral vector or plasmid containing structural and functional genetic elements, or portions thereof, that are primarily derived from a gammaretrovirus. A lentiviral vector can comprise a viral vector or plasmid containing structural and functional genetic elements, or portions thereof, including LTRs that are primarily derived from a lentivirus.

In a first aspect, the present invention relates to an expression vector, an expression cassette or a pseudotyped viral vector particle comprising at least one nucleic acid encoding at least one Koala Retrovirus (KoRV) Envelope glycoprotein (Env).

The nucleic acid, which is contained the expression vector, the expression cassette or the pseudotyped viral vector particle of the first aspect of the invention may further encode at least one KoRV Env which lacks the C-terminal R-peptide or part of the R-peptide. In general, the term “C-terminal R-peptide”, as used herein, means any nucleic acid sequence encoding the utmost C-terminus of the KoRV Env or any nucleic acid sequence C-terminal of the nucleic acid sequence encoding the transmembrane domain. For example, in the case of KoRVA, the C-terminal R-peptide corresponds to amino acid positions 645-659 of KoRVA. The full-length protein KoRVA sequence is shown in SEQ ID No: 6. The C-terminal R-peptide may therefore be any peptide region in any KoRV protein corresponding to that region of KORVA. The R peptide can be determined by analysis of sequence motifs. For example, in the case of KoRVB, the C-terminal R-peptide corresponds to amino acid positions 652 to 666 of KoRVB. The full-length protein KoRVA sequence is shown in SEQ ID No: 8.

The KoRV Env protein sequences shown in SEQ ID Nos: 2, 4, 6 and 8 include the N-terminal signal peptide.

In the pseudotyped viral vector particles of the invention, the signal peptide is removed from KoRV Env proteins, thereby generating mature KoRV Env proteins. The pseudotyped viral vector particles of the invention comprise mature KoRVA Env proteins. Preferably, the mature KoRVA Env protein lacks the R-peptide or lacks part of the R-peptide. Alternatively, the mature KoRVA Env protein comprises the complete cytoplasmic domain.

According to the present invention, “mature KoRV Env” is understood as a KoRV Env protein lacking the N-terminal signal peptide.

In the pseudotyped viral vector particles of the invention, the signal peptide is removed from KoRV Env proteins. The pseudotyped viral vector particles of the invention comprise mature KoRV Env proteins. Preferably, the mature KoRV Env protein lacks the R-peptide or lacks part of the R-peptide. Alternatively, the mature KORV Env protein comprises the complete cytoplasmic domain.

Further, preferably, the nucleic acid of the invention as comprised in the expression vector, expression cassette or pseudotyped viral vector particle of the invention, encoding at least one KoRV Env lacking the C-terminal R-peptide, may lack the complete C-terminal R-peptide or may lack part of the C-terminal R-peptide and may, therefore, be of variable length. If the nucleic acid as contained in the expression vector, expression cassette or pseudotyped viral vector particle lacks part of the C-terminal R-peptide, it may lack, one, two, three, or more sequence portions thereof and/or these sequence portions may be contiguous or separate.

The nucleic acid encoding the at least one KoRV Env which lacks the C-terminal R-peptide may be prepared by, e.g. introducing the nucleic acid already lacking the nucleic acid sequence encoding the C-terminal R-peptide into an expression vector or an expression cassette. Moreover, the nucleic acid encoding the at least one KORV Env which lacks the C-terminal R-peptide may be prepared by introducing full-length KoRV Env in an expression vector or the expression cassette and enzymatically cutting out the nucleic acid sequence encoding the C-terminal R-peptide.

In a preferred embodiment, the at least one nucleic acid encodes at least one KoRV Env which lacks the C-terminal R-peptide.

In a second aspect, the present invention relates to a nucleic acid encoding at least one KoRV Env which lacks the fusion inhibitory R-peptide (R-peptide) or lacks part of the R-peptide.

Such nucleic acids encoding at least one KORV Env which lacks the fusion inhibitory R-peptide (R-peptide) or lacks parts of the R-peptide do not naturally occur in nature. Moreover, such nucleic acids are not disclosed in the art.

The nucleic acid according to the second aspect of the present invention generally relates to any nucleotide molecule which encodes the at least one KoRV Env which lacks the fusion inhibitory R-peptide (R-peptide) or lacks part of the R-peptide and which may be of variable length. Examples of a nucleic acid according to the second aspect of the invention include, but are not limited to, plasmids, vectors, or any kind of DNA and/or RNA fragment(s) which can be isolated by standard molecular biology procedures, including, e.g. ion-exchange chromatography. A nucleic acid according to the second aspect of the invention may be used for transfection or transduction of a particular cell or organism.

Nucleic acids encoding a KoRVA Env lacking the R-peptide, nucleic acids encoding a KoRVA Env lacking the R-peptide and combinations of nucleic acids encoding a KoRVA Env lacking the R-peptide and nucleic acids encoding a KoRVB Env lacking the R-peptide were used successfully in Examples 1 and 2 for pseudotyped viral vector particle production and subsequent, efficient transduction of NK cells with a payload nucleic acid.

KORV Env sequences and nucleic acid sequences encoding KoRV Env sequences which can be used according to the invention are shown in Table 1 below.

TABLE 1

| SEQ | | |
|---|---|---|
| ID | | |
| No: | Sequence | Description |
| 1 | ATGCTTCTCATCTCAAACCCGCGCCACCTG | Encodes |
| | GGGCACCCGATGAGTCCTGGGAACTGGAAA | KoRVA Env |
| | AGACTGATCATCCTCTTAAGTTGCGTGTTC | lacking the |
| | GGCGGCGCCGAAATGAATCAACAGCATAAC | R-peptide |
| | AACCCTCACCAACCCATGACTCTCACCTGG | |
| | CAGGTACTGTCCCAGACGGGAAGTGTCGTT | |
| | TGGGAAAAGAAAGCAGTCGAGCCACCCTGG | |
| | ACGTGGTGGCCCTCTCTTGAGCCTGATGTG | |
| | TGCGCGCTGGTAGCCGGTCTTGAGTCCTGG | |
| | GACATCCCGGAACTCACTGCATCGGCCTCT | |
| | CAACAGGCCAGGCCCCCTGATTCAAACTAT | |
| | GAACATGCTTATAATCAGATCACTTGGGGA | |
| | ACCCTGGGATGTAGCTATCCCCGGGCCAGA | |
| | ACAAGAATTGCTAGGTCCCAGTTCTACGTG | |
| | TGCCCCCGGGATGGCCGTTCCCTGTCCGAG | |
| | GCCCGGAGGTGCGGAGGACTTGAATCTTTG | |
| | TACTGTAAAGAGTGGGGTTGTGAGACCGCG | |
| | GGAACCGCCTACTGGCAACCCCGGTCCTCG | |
| | TGGGACCTTATAACTGTAGGCCAGGGTCAC | |
| | CCTACAGGGACATGCGAGCGCACCGGTTGG | |
| | TGCAACCCACTCAAGATAGAGTTCACCGAG | |
| | CCTGGCAAACGGTTCCGAAACTGGCTACAG | |
| | GGGCGAACCTGGGGTCTAAGGTTCTATGTG | |
| | ACTGGACACCCAGGCGTGCAGTTGACCATA | |
| | CGACTAGTGATCACAAGTCCCCCACCGGTA | |
| | GTAGTGGGCCCCGATCCTGTCCTCGCAGAA | |
| | CAAGGACCTCCCAGAAAAATTCCCTTCCTC | |
| | CCCAGAGTGCCGGTTCCTACCCTATCACCC | |
| | CCAGCGTCCCCTATCCCCACGGTACAGGCC | |
| | AGCCCCCCGGCCCCTAGTACTCCATCTCCC | |
| | ACCACGGGCGACCGGCTCTTTGGCCTCGTA | |
| | CAAGGGGCCTTTCTGGCCTTGAATGCCACC | |
| | AACCCGGAGGCCACGGAGTCTTGCTGGCTC | |
| | TGTCTAGCCCTCGGCCCTCCTTATTACGAA | |
| | GGAATCGCAACTCCAGGGCAAGTCACTTAT | |
| | GCCTCCACAGATTCCCAATGTCGCTGGGGA | |
| | GGAAAAGGGAAGCTCACCCTCACTGAAGTC | |
| | TCTGGACTCGGGTTATGTATAGGGAAAGTA | |
| | CCTCCTACTCACCAACATCTCTGCAACCTG | |
| | ACTATACCCTTAAACGCCTCACATACTCAC | |
| | AAGTACCTCCTCCCCTCCAACCGCAGCTGG | |
| | TGGGCTTGTAATTCTGGCCTCACCCCCTGC | |
| | CTCTCTACATCAGTCTTCAACCAGTCTAAC | |
| | GATTTCTGTATCCAGATCCAGCTTGTCCCT | |
| | CGCATCTACTATCACCCAGACGGTACCTTG | |
| | CTACAGGCCTATGAGTCCCCCCACTCAAGA | |
| | AACAAGAGAGAGCCTGTCTCACTCACCCTG | |
| | GCTGTCCTTCTCGGATTAGGGGTCGCAGCA | |
| | GGTATAGGTACCGGCTCGACCGCCCTAATA | |
| | AAAGGGCCCATAGACCTCCAACAAGGTTTG | |
| | ACTAGCCTCCAGATTGCCATGGATACAGAC | |
| | CTTAGGGCCCTTCAAGACTCCATAAGTAAA | |
| | CTAGAGGATTCCTTAACCTCCCTGTCTGAA | |
| | GTAGTGCTCCAGAATAGGAGAGGCCTTGAT | |
| | CTGCTATTTTTGAAGGAAGGGGGCCTTTGT | |
| | GCAGCCCTAAAAGAGGAATGCTGTTTCTAT | |
| | GTTGACCACTCAGGCGCGGTGCGAGACTCC | |
| | ATGAGGAGACTCAAGGAAAGGTTAGATAAG | |
| | AGGCAGTTAGAGCACCAAAAGAATTTAAGT | |
| | TGGTACGAGGGATGGTTCAACCGTTCCCCC | |
| | TGGCTTACTACTTTACTGTCTGCCCTTGCT | |
| | GGTCCCCTGCTACTCCTCCTTCTGTTACTC | |
| | ACCCTCGGGCCTTGTGTCATCAATAAGTTA | |
| | GTGCAATTCATCAATGATAGGGTTAGTGCA | |
| | GTAAGGATTCTGTAA | |
| 2 | MLLISNPRHLGHPMSPGNWKRLIILLSCVF | KoRVA Env |
| | GGAEMNQQHNNPHQPMTLTWQVLSQTGSVV | lacking the |
| | WEKKAVEPPWTWWPSLEPDVCALVAGLESW | R-peptide |
| | DIPELTASASQQARPPDSNYEHAYNQITWG | Sequence |
| | TLGCSYPRARTRIARSQFYVCPRDGRSLSE | includes the |
| | ARRCGGLESLYCKEWGCETAGTAYWQPRSS | signal |
| | WDLITVGQGHPTGTCERTGWCNPLKIEFTE | peptide |
| | PGKRFRNWLQGRTWGLRFYVTGHPGVQLTI | |
| | RLVITSPPPVVVGPDPVLAEQGPPRKIPFL | |
| | PRVPVPTLSPPASPIPTVQASPPAPSTPSP | |
| | TTGDRLFGLVQGAFLALNATNPEATESCWL | |
| | CLALGPPYYEGIATPGQVTYASTDSQCRWG | |
| | GKGKLTLTEVSGLGLCIGKVPPTHQHLCNL | |
| | TIPLNASHTHKYLLPSNRSWWACNSGLTPC | |
| | LSTSVFNQSNDFCIQIQLVPRIYYHPDGTL | |
| | LQAYESPHSRNKREPVSLTLAVLLGLGVAA | |
| | GIGTGSTALIKGPIDLQQGLTSLQIAMDTD | |
| | LRALQDSISKLEDSLTSLSEVVLQNRRGLD | |
| | LLFLKEGGLCAALKEECCFYVDHSGAVRDS | |
| | MRRLKERLDKRQLEHQKNLSWYEGWFNRSP | |
| | WLTTLLSALAGPLLLLLLLLTLGPCVINKL | |
| | VQFINDRVSAVRIL* | |
| 3 | ATGCTTCTCATCTCAAACCCGCGCCACCTG | Encodes |
| | GGGCACCCGATGAGTCCTGGGAACTGGAAA | KoRVB Env |
| | AGACTGATCATCCTCTTAAGTTGCGTGTTC | lacking the |
| | GGCGGCGCCGAAATGAATCAACAGCATAAC | R-peptide |
| | AACCCTCACCAACCCATGACTCTCACCTGG | |
| | CAGGTACTGTCCCAGACGGGAAGTGTCGTT | |
| | TGGGAAAAGAAAGCAGTCGAGCCACCCTGG | |
| | ACGTGGTGGCCCTCTCTTGAGCCTGATGTG | |
| | TGTGCGCTGGCGGCGGGTCTTGAGACCTGG | |
| | GGCATCCCGCACCTCACCGCGCCAGAATCT | |
| | CAACAGTCTGCGCCCCCCGATGGCTGTGGG | |
| | GTGCTTTATAGTCAGGTCGGCAGAGGGTTG | |
| | GTCAGGCAATCGAGCTATGGAACCTTGGGG | |
| | TGTCGCTGTCCCCGAGACCGGAACAGACTG | |
| | GCTCAATCTCAGTTCTATGTGTGTCCCCGA | |
| | GATGGCCGTTCCCTGTCCGAGGCCTGGAGG | |
| | TGCGGAGGGTTTGAGTCTTTGTATTGTAAA | |
| | GAGTGGGGTTGTGAGACCACGGGAACCGCC | |
| | TACTGGCAACCCCGGTCCTCGTGGGACCTT | |
| | ATAACTGTAGGCCTGGGCCACCCTACGGGG | |
| | ACATGCGAACACACCGGTTGGTGCAACCCA | |
| | CTCAAGATAGAGTTCACCGAGCCTGGCAAA | |
| | CGGTTCCGAAACTGGCTACAGGGGCGAACC | |
| | TGGGGTCTAAGGTTCTATGTGACTGGACAC | |
| | CCAGGCGTGCAGTTGACCATACGACTAGTG | |
| | ATCACAAGTCCCCCACCGGTAGTAGTGGGC | |
| | CCCGATCCTGTCCTCGCAGAACAAGGACCT | |
| | CCCAGAAAAATTCCCTTCCTCCCCAGAGTG | |
| | CCGGTTCCTACCCTATCACCCCCAGCGTCC | |
| | CCTATCCCCACGGTACAGGCAAGCCCCCCG | |
| | GCCCCTAGTACTCCATCTCCCACCACGGGC | |
| | GACCGGCTCTTTGGCCTCGTACAAGGGGCC | |
| | TTTCTGGCCTTGAATGCCACCAACCCGGAG | |
| | GCCACGGAGTCTTGCTGGCTCTGTCTAGCC | |
| | CTCGGCCCTCCTTATTACGAAGGAATCGCA | |
| | ACTCCAGGGCAAGTCACTTATGCCTCCACA | |
| | GATTCCCAATGTCGCTGGGGAGGAAAAGGG | |
| | AAGCTCACCCTCACTGAAGTCTCTGGACTC | |
| | GGGTTATGTATAGGGAAAGTACCTCCTACT | |
| | CACCAACATCTCTGCAACCTGACTATACCC | |
| | TTAAACGCCTCACATACTCACAAGTACCTC | |
| | CTCCCCTCCAACCACAGCTGGTGGGCTTGT | |
| | AATTCTGGCCTCACCCCCTGCCTCTCTACA | |
| | TCAGTCTTCAACCAGTCTAACGATTTCTGT | |
| | ATCCAGATCCAGCTTGTCCCTCGCATCTAC | |
| | TATCACCCAGACGGTACCTTGCTACAGGCC | |
| | TATGAGTCCCCCCACCCTAGAAACAAGAGA | |
| | GAGCCTGTCTCACTCACCCTGGCTGTCCTT | |
| | CTCGGATTAGGGGTCGCAGCAGGTATAGGT | |
| | ACCGGCTCGACCGCCCTAATAAAAGGGCCC | |
| | ATAGACCTCCAACAAGGTTTGACTAGCCTC | |
| | CAAATTGCCATGGATACAGACCTTAGAGCC | |
| | CTTCAAGACTCCGTAAGTAAACTAGAGAAC | |
| | TCATTAACCTCCCTTTCTGAAGTAGTGCTC | |
| | CAGAATAGGAGAGGCCTTGATCTGCTATTT | |
| | TTGAAGGAAGGGGGCCTTTGTGCAGCCCTA | |
| | AAAGAGGAGTGCTGTTTCTATGTTGACCAC | |
| | TCAGGCGCGGTGCGAGACTCCATGAGGAGA | |
| | CTCAAGGAAAGGTTAGATAAGAGGCAGTTA | |
| | GAGCACCAAAAGAATTTAAGTTGGTACGAG | |
| | GGATGGTTCAACCGTTCCCCCTGGCTTACT | |
| | ACTTTACTGTCTGCCCTTGCTGGTCCCCTG | |
| | CTACTCCTCCTTCTGTTACTCACCCTCGGG | |
| | CCTTGTGTCATCAATAAGTTAGTGCAATTC | |
| | ATCAATGATAGGGTTAGTGCAGTAAGGATT | |
| | CTGTAA | |
| 4 | MLLISNPRHLGHPMSPGNWKRLIILLSCVF | KoRVB Env |
| | GGAEMNQQHNNPHQPMTLTWQVLSQTGSVV | lacking the |
| | WEKKAVEPPWTWWPSLEPDVCALAAGLETW | R-peptide |
| | GIPHLTAPESQQSAPPDGCGVLYSQVGRGL | Sequence |
| | VRQSSYGTLGCRCPRDRNRLAQSQFYVCPR | includes the |
| | DGRSLSEAWRCGGFESLYCKEWGCETTGTA | signal |
| | YWQPRSSWDLITVGLGHPTGTCEHTGWCNP | peptide |
| | LKIEFTEPGKRFRNWLQGRTWGLRFYVTGH | |
| | PGVQLTIRLVITSPPPVVVGPDPVLAEQGP | |
| | PRKIPFLPRVPVPTLSPPASPIPTVQASPP | |
| | APSTPSPTTGDRLFGLVQGAFLALNATNPE | |
| | ATESCWLCLALGPPYYEGIATPGQVTYAST | |
| | DSQCRWGGKGKLTLTEVSGLGLCIGKVPPT | |
| | HQHLCNLTIPLNASHTHKYLLPSNHSWWAC | |
| | NSGLTPCLSTSVFNQSNDFCIQIQLVPRIY | |
| | YHPDGTLLQAYESPHPRNKREPVSLTLAVL | |
| | LGLGVAAGIGTGSTALIKGPIDLQQGLTSL | |
| | QIAMDTDLRALQDSVSKLENSLTSLSEVVL | |
| | QNRRGLDLLFLKEGGLCAALKEECCFYVDH | |
| | SGAVRDSMRRLKERLDKRQLEHQKNLSWYE | |
| | GWFNRSPWLTTLLSALAGPLLLLLLLLTLG | |
| | PCVINKLVQFINDRVSAVRIL* | |
| 5 | atgcttctcatctcaaacccgcgccacctg | Encodes |
| | gggcacccgatgagtcctgggaactggaaa | full-length |
| | agactgatcatcctcttaagttgcgtgttc | KoRVA Env |
| | ggcggcgccgaaatgaatcaacagcataac | |
| | aaccctcaccaacccatgactctcacctgg | |
| | caggtactgtcccagacgggaagtgtcgtt | |
| | tgggaaaagaaagcagtcgagccaccctgg | |
| | acgtggtggccctctcttgagcctgatgtg | |
| | tgcgcgctggtagccggtcttgagtcctgg | |
| | gacatcccggaactcactgcatcggcctct | |
| | caacaggccaggccccctgattcaaactat | |
| | gaacatgcttataatcagatcacttgggga | |
| | accctgggatgtagctatccccgggccaga | |
| | acaagaattgctaggtcccagttctacgtg | |
| | tgcccccgggatggccgttccctgtccgag | |
| | gcccggaggtgcggaggacttgaatctttg | |
| | tactgtaaagagtggggttgtgagaccgcg | |
| | ggaaccgcctactggcaaccccggtcctcg | |
| | tgggaccttataactgtaggccagggtcac | |
| | cctacagggacatgcgagcgcaccggttgg | |
| | tgcaacccactcaagatagagttcaccgag | |
| | cctggcaaacggttccgaaactggctacag | |
| | gggcgaacctggggtctaaggttctatgtg | |
| | actggacacccaggcgtgcagttgaccata | |
| | cgactagtgatcacaagtcccccaccggta | |
| | gtagtgggccccgatcctgtcctcgcagaa | |
| | caaggacctcccagaaaaattcccttcctc | |
| | cccagagtgccggttcctaccctatcaccc | |
| | ccagcgtcccctatccccacggtacaggct | |
| | agccccccggcccctagtactccatctccc | |
| | accacgggcgaccggctctttggcctcgta | |
| | caaggggcctttctggccttgaatgccacc | |
| | aacccggaggccacggagtcttgctggctc | |
| | tgtctagccctcggccctccttattacgaa | |
| | ggaatcgcaactccagggcaagtcacttat | |
| | gcctccacagattcccaatgtcgctgggga | |
| | ggaaaagggaagctcaccctcactgaagtc | |
| | tctggactcgggttatgtatagggaaagta | |
| | cctcctactcaccaacatctctgcaacctg | |
| | actatacccttaaacgcctcacatactcac | |
| | aagtacctcctcccctccaaccgcagctgg | |
| | tgggcttgtaattctggcctcaccccctgc | |
| | ctctctacatcagtcttcaaccagtctaac | |
| | gatttctgtatccagatccagcttgtccct | |
| | cgcatctactatcacccagacggtaccttg | |
| | ctacaggcctatgagtccccccactctaga | |
| | aacaagagagagcctgtctcactcaccctg | |
| | gctgtccttctcggattaggggtcgcagca | |
| | ggtataggtaccggctcgaccgccctaata | |
| | aaagggcccatagacctccaacaaggtttg | |
| | actagcctccagattgccatggatacagac | |
| | cttagggcccttcaagactccataagtaaa | |
| | ctagaggattccttaacctccctgtctgaa | |
| | gtagtgctccagaataggagaggccttgat | |
| | ctgctatttttgaaggaagggggcctttgt | |
| | gcagccctaaaagaggaatgctgtttctat | |
| | gttgaccactcaggcgcggtgcgagactcc | |
| | atgaggagactcaaggaaaggttagataag | |
| | aggcagttagagcaccaaaagaatttaagt | |
| | tggtacgagggatggttcaaccgttccccc | |
| | tggcttactactttactgtctgcccttgct | |
| | ggtcccctgctactcctccttctgttactc | |
| | accctcgggccttgtgtcatcaataagtta | |
| | gtgcaattcatcaatgatagggttagtgca | |
| | gtaaggattctggttctcaggcacaagtac | |
| | cagaccctagacaacgaggataacctttaa | |
| 6 | MLLISNPRHLGHPMSPGNWKRLIILLSCVF | Full-length |
| | GGAEMNQQHNNPHQPMTLTWQVLSQTGSVV | KoRVA Env |
| | WEKKAVEPPWTWWPSLEPDVCALVAGLESW | Sequence |
| | DIPELTASASQQARPPDSNYEHAYNQITWG | includes the |
| | TLGCSYPRARTRIARSQFYVCPRDGRSLSE | signal |
| | ARRCGGLESLYCKEWGCETAGTAYWQPRSS | peptide |
| | WDLITVGQGHPTGTCERTGWCNPLKIEFTE | |
| | PGKRFRNWLQGRTWGLRFYVTGHPGVQLTI | |
| | RLVITSPPPVVVGPDPVLAEQGPPRKIPFL | |
| | PRVPVPTLSPPASPIPTVQASPPAPSTPSP | |
| | TTGDRLFGLVQGAFLALNATNPEATESCWL | |
| | CLALGPPYYEGIATPGQVTYASTDSQCRWG | |
| | GKGKLTLTEVSGLGLCIGKVPPTHQHLCNL | |
| | TIPLNASHTHKYLLPSNRSWWACNSGLTPC | |
| | LSTSVFNQSNDFCIQIQLVPRIYYHPDGTL | |
| | LQAYESPHSRNKREPVSLTLAVLLGLGVAA | |
| | GIGTGSTALIKGPIDLQQGLTSLQIAMDTD | |
| | LRALQDSISKLEDSLTSLSEVVLQNRRGLD | |
| | LLFLKEGGLCAALKEECCFYVDHSGAVRDS | |
| | MRRLKERLDKRQLEHQKNLSWYEGWFNRSP | |
| | WLTTLLSALAGPLLLLLLLLTLGPCVINKL | |
| | VQFINDRVSAVRILVLRHKYQTLDNEDNL* | |
| 7 | ATGCTTCTCATCTCAAACCCGCGCCACCTG | Encodes |
| | GGGCACCCGATGAGTCCTGGGAACTGGAAA | full-length |
| | AGACTGATCATCCTCTTAAGTTGCGTGTTC | KoRVB Env |
| | GGCGGCGCCGAAATGAATCAACAGCATAAC | |
| | AACCCTCACCAACCCATGACTCTCACCTGG | |
| | CAGGTACTGTCCCAGACGGGAAGTGTCGTT | |
| | TGGGAAAAGAAAGCAGTCGAGCCACCCTGG | |
| | ACGTGGTGGCCCTCTCTTGAGCCTGATGTG | |
| | TGTGCGCTGGCGGCGGGTCTTGAGACCTGG | |
| | GGCATCCCGCACCTCACCGCGCCAGAATCT | |
| | CAACAGTCTGCGCCCCCCGATGGCTGTGGG | |
| | GTGCTTTATAGTCAGGTCGGCAGAGGGTTG | |
| | GTCAGGCAATCGAGCTATGGAACCTTGGGG | |
| | TGTCGCTGTCCCCGAGACCGGAACAGACTG | |
| | GCTCAATCTCAGTTCTATGTGTGTCCCCGA | |
| | GATGGCCGTTCCCTGTCCGAGGCCTGGAGG | |
| | TGCGGAGGGTTTGAGTCTTTGTATTGTAAA | |
| | GAGTGGGGTTGTGAGACCACGGGAACCGCC | |
| | TACTGGCAACCCCGGTCCTCGTGGGACCTT | |
| | ATAACTGTAGGCCTGGGCCACCCTACGGGG | |
| | ACATGCGAACACACCGGTTGGTGCAACCCA | |
| | CTCAAGATAGAGTTCACCGAGCCTGGCAAA | |
| | CGGTTCCGAAACTGGCTACAGGGGCGAACC | |
| | TGGGGTCTAAGGTTCTATGTGACTGGACAC | |
| | CCAGGCGTGCAGTTGACCATACGACTAGTG | |
| | ATCACAAGTCCCCCACCGGTAGTAGTGGGC | |
| | CCCGATCCTGTCCTCGCAGAACAAGGACCT | |
| | CCCAGAAAAATTCCCTTCCTCCCCAGAGTG | |
| | CCGGTTCCTACCCTATCACCCCCAGCGTCC | |
| | CCTATCCCCACGGTACAGGCTAGCCCCCCG | |
| | GCCCCTAGTACTCCATCTCCCACCACGGGC | |
| | GACCGGCTCTTTGGCCTCGTACAAGGGGCC | |
| | TTTCTGGCCTTGAATGCCACCAACCCGGAG | |
| | GCCACGGAGTCTTGCTGGCTCTGTCTAGCC | |
| | CTCGGCCCTCCTTATTACGAAGGAATCGCA | |
| | ACTCCAGGGCAAGTCACTTATGCCTCCACA | |
| | GATTCCCAATGTCGCTGGGGAGGAAAAGGG | |
| | AAGCTCACCCTCACTGAAGTCTCTGGACTC | |
| | GGGTTATGTATAGGGAAAGTACCTCCTACT | |
| | CACCAACATCTCTGCAACCTGACTATACCC | |
| | TTAAACGCCTCACATACTCACAAGTACCTC | |
| | CTCCCCTCCAACCACAGCTGGTGGGCTTGT | |
| | AATTCTGGCCTCACCCCCTGCCTCTCTACA | |
| | TCAGTCTTCAACCAGTCTAACGATTTCTGT | |
| | ATCCAGATCCAGCTTGTCCCTCGCATCTAC | |
| | TATCACCCAGACGGTACCTTGCTACAGGCC | |
| | TATGAGTCCCCCCACCCTAGAAACAAGAGA | |
| | GAGCCTGTCTCACTCACCCTGGCTGTCCTT | |
| | CTCGGATTAGGGGTCGCAGCAGGTATAGGT | |
| | ACCGGCTCGACCGCCCTAATAAAAGGGCCC | |
| | ATAGACCTCCAACAAGGTTTGACTAGCCTC | |
| | CAAATTGCCATGGATACAGACCTTAGAGCC | |
| | CTTCAAGACTCCGTAAGTAAACTAGAGAAT | |
| | TCCTTAACCTCCCTTTCTGAAGTAGTGCTC | |
| | CAGAATAGGAGAGGCCTTGATCTGCTATTT | |
| | TTGAAGGAAGGGGGCCTTTGTGCAGCCCTA | |
| | AAAGAGGAGTGCTGTTTCTATGTTGACCAC | |
| | TCAGGCGCGGTGCGAGACTCCATGAGGAGA | |
| | CTCAAGGAAAGGTTAGATAAGAGGCAGTTA | |
| | GAGCACCAAAAGAATTTAAGTTGGTACGAG | |
| | GGATGGTTCAACCGTTCCCCCTGGCTTACT | |
| | ACTTTACTGTCTGCCCTTGCTGGTCCCCTG | |
| | CTACTCCTCCTTCTGTTACTCACCCTCGGG | |
| | CCTTGTGTCATCAATAAGTTAGTGCAATTC | |
| | ATCAATGATAGGGTTAGTGCAGTAAGGATT | |
| | CTGGTTCTCAGGCACAAGTACCAGACCCTA | |
| | GACAACGAGGATAACCTTTAA | |
| 8 | MLLISNPRHLGHPMSPGNWKRLIILLSCVF | full-length |
| | GGAEMNQQHNNPHQPMTLTWQVLSQTGSVV | KoRVB Env |
| | WEKKAVEPPWTWWPSLEPDVCALAAGLETW | Sequence |
| | GIPHLTAPESQQSAPPDGCGVLYSQVGRGL | includes the |
| | VRQSSYGTLGCRCPRDRNRLAQSQFYVCPR | signal |
| | DGRSLSEAWRCGGFESLYCKEWGCETTGTA | peptide |
| | YWQPRSSWDLITVGLGHPTGTCEHTGWCNP | |
| | LKIEFTEPGKRFRNWLQGRTWGLRFYVTGH | |
| | PGVQLTIRLVITSPPPVVVGPDPVLAEQGP | |
| | PRKIPFLPRVPVPTLSPPASPIPTVQASPP | |
| | APSTPSPTTGDRLFGLVQGAFLALNATNPE | |
| | ATESCWLCLALGPPYYEGIATPGQVTYAST | |
| | DSQCRWGGKGKLTLTEVSGLGLCIGKVPPT | |
| | HQHLCNLTIPLNASHTHKYLLPSNHSWWAC | |
| | NSGLTPCLSTSVFNQSNDFCIQIQLVPRIY | |
| | YHPDGTLLQAYESPHPRNKREPVSLTLAVL | |
| | LGLGVAAGIGTGSTALIKGPIDLQQGLTSL | |
| | QIAMDTDLRALQDSVSKLENSLTSLSEVVL | |
| | QNRRGLDLLFLKEGGLCAALKEECCFYVDH | |
| | SGAVRDSMRRLKERLDKRQLEHQKNLSWYE | |
| | GWFNRSPWLTTLLSALAGPLLLLLLLLTLG | |
| | PCVINKLVQFINDRVSAVRILVLRHKYQTL | |
| | DNEDNL* | |

The nucleic acid molecule according to the second aspect of the present invention may be in the form of RNA, such as mRNA or cRNA, or in the form of DNA, including, for instance, cDNA and genomic DNA e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand. Nucleic acid molecule according to the second aspect of the invention may also refer to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA.

Additionally, the nucleic acid may contain one or more modified bases. Such nucleic acids may also contain modifications e.g. in the ribose-phosphate backbone to increase stability and half-life of such molecules in physiological environments. Thus, DNAs or RNAs with backbones modified for stability or for other reasons are “nucleic acid molecule” as that feature is intended herein. Moreover, DNAs or RNAs comprising unusual bases, such as inosine, or modified bases, such as tritylated bases, to name just two examples, are nucleic acid molecule within the context of the present invention. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia.

Furthermore, the nucleic acid molecule according to the second aspect of the invention encoding the nucleic acid encoding at least one KoRV Env which lacks the fusion inhibitory R-peptide (R-peptide) or lacks part of the R-peptide can be functionally linked, using standard techniques such as standard cloning techniques, to any desired sequence, such as a regulatory sequence, leader sequence, heterologous marker sequence or a heterologous coding sequence, e.g. to create a fusion protein.

The nucleic acid of the second aspect of the invention may be originally formed in vitro or in a cell in culture, in general, by the manipulation of nucleic acids by endonucleases and/or exonucleases and/or polymerases and/or ligases and/or recombinases or other methods known to the skilled practitioner to produce the nucleic acids.

The nucleic acid of second aspect of the invention may be comprised in an expression vector, an expression cassette or a pseudotyped viral vector particle, wherein the nucleic acid is operably linked to a promoter sequence capable of promoting the expression of the nucleic acid in a host cell.

The nucleic acid according to the second aspect encoding at least one KoRV Env which lacks the fusion inhibitory R-peptide (R-peptide) or lacks part of the R-peptide may also encode at least two KoRV Env which lack the fusion inhibitory R-peptide (R-peptide) or lack part of the R-peptide, at least three KoRV Env which lack the fusion inhibitory R-peptide (R-peptide) or lack part of the R-peptide or at least four KORV Env which lack the fusion inhibitory R-peptide (R-peptide) or lack part of the R-peptide.

In the first and second aspect, the at least one KoRV Env may be selected from any KORV Env of any KoRV Env species. For example, the KoRV Env may be selected from KoRVA Env, KoRVB Env, KoRVC Env, KoRVD Env, KoRVE Env, KoRVF Env, KORVJ Env and any combination thereof. Preferably, the at least one KoRV Env may be selected from KoRVA, KoRVB and a combination or mixture thereof.

In a preferred embodiment of the first and second aspect, the at least one KoRV Env is selected from KoRVA, KoRVB and a combination thereof.

Moreover, the sequence of KoRVA Env may be selected from (a) SEQ ID No: 2, (b) SEQ ID No: 6, wherein SEQ ID No: 6 is optionally C-terminally truncated for one or more of amino acids 645 to 659 of SEQ No: No: 6, and (c) a sequence of (a) or (b) lacking the signal peptide.

In case of KoRVA, the signal peptide corresponds to amino acid positions 1-35 (see FIG. 1). Alternatively, a KoRV Env is encompassed wherein only part of the signal peptide is deleted. For example, amino acids 1-33 or 1-34 may be deleted.

Moreover, if the sequence of the KoRVA Env is selected from (b) SEQ ID No: 6, wherein SEQ ID No: 6 is C-terminally truncated for one or more of amino acids 645 to 659 of SEQ No: No: 6, the C-terminus of SEQ ID No: 6 may also be terminally truncated for two or more of amino acids 645 to 659 of SEQ No: 6, three or more of amino acids 645 to 659 of SEQ No: 6, five or more amino acids 645 to 659 of SEQ No: 6 or 10 or more amino acids 645 to 659 of SEQ No: 6. For example, the C-terminus of SEQ ID No: 6 may be terminally truncated for one, two, three, four, five, six, seven, eight, nine, ten, eleven, twelve, thirteen, fourteen or fifteen of amino acids 645 to 659 of SEQ No: 6.

Further, the sequence of the KoRVB Env may be selected from (a) SEQ ID No: 4, (b) SEQ ID No: 8, wherein SEQ ID No: 8 is optionally C-terminally truncated for one or more of amino acids 652 to 666 of SEQ No: No: 8, and (c) a sequence of (a) or (b) lacking the signal peptide.

In case of KoRVB, the signal peptide corresponds to amino acid positions 1-35. Alternatively, a KoRV Env is encompassed wherein only part of the signal peptide is deleted. For example, amino acids 1-33 or 1-34 may be deleted.

Moreover, if the sequence of the KoRVB Env is selected from (b) SEQ ID No: 8, wherein SEQ ID No: 8 is C-terminally truncated for one or more of amino acids 652 to 666 of SEQ No: 8, the C-terminus of SEQ ID No: 8 may also be terminally truncated for two or more of amino acids 652 to 666 of SEQ No: 8, three or more of amino acids 652 to 666 of SEQ No: 8, five or more amino acids 652 to 666 of SEQ No: 8 or 10 or more amino acids 652 to 666 of SEQ No: 8. For example, the C-terminus of SEQ ID No: 8 may be terminally truncated for one, two, three, four, five, six, seven, eight, nine, ten, eleven, twelve, thirteen, fourteen or fifteen of amino acids 652 to 666 of SEQ No: 8.

The sequence of the at least one nucleic acid encoding at least one KoRV Env may comprise or consist of one or more of SEQ ID No: 1, SEQ ID No: 3, SEQ ID No: 5, wherein SEQ ID No: 5 is optionally truncated at the 3′ end for one or more of the 48 3′ terminal nucleotides, SEQ ID No: 7, wherein SEQ ID No: 7 is optionally truncated at the 3′ end for one or more of the 48 3′ terminal nucleotides, or a variant thereof comprising one or more silent mutations.

If the sequence of the at least one nucleic acid encoding at least one KoRV Env comprises or consists of one or more of SEQ ID No: 1, SEQ ID No: 3, SEQ ID No: 5, it may comprise or consist of any combination of these sequences, i.e. it may comprise or consist of SEQ ID No: 1; SEQ ID No: 3; SEQ ID No: 5; SEQ ID No: 1 and SEQ ID No: 3; SEQ ID No: 1 and SEQ ID No: 5; SEQ ID No: 3 and SEQ ID No: 5; or SEQ ID No: 1, SEQ ID No: 3 and SEQ ID No: 5. Further, it may also mean that it comprises or consists of these sequences in any order. Moreover, if the at least one nucleic acid encoding at least one KoRV Env comprises or consists of two or more of SEQ ID No: 1, SEQ ID No: 3, SEQ ID No: 5, this may also mean that it comprises or consists of any one of SEQ ID No: 1, SEQ ID No: 3, SEQ ID No: 5 twice.

If SEQ ID No: 5 is truncated at the 3′ end for one or more of the 48 3′ terminal nucleotides, this means that SEQ ID No: 5 may be truncated at the 3′ end for one to 48 of the 48 3′ terminal nucleotides, preferably for 2 to 24 of the 48 3′ terminal nucleotides, more preferably for 4 to 12 of the 48 3′ terminal nucleotides. If SEQ ID No: 5 is truncated at the 3′ end for two or more of the 48 3′ terminal nucleotides, the truncated nucleotides may be adjacent or separated by nucleotides that are not part of the truncation. The same of above features of the SEQ ID No: 5 truncated at the 3′ end for one or more of the 48 3′ terminal nucleotides apply, if SEQ ID No: 7 is truncated at the 3′ end for one or more of the 48 3′ terminal nucleotides.

The term “silent mutation”, as used herein, describes any nucleotide mutation not having any effect on the protein encoded by such nucleic acid. For example, a silent mutation may not affect the function or folding of the protein encoded by such nucleic acid. In a further example, a silent mutation may not have an effect on the synthesis of the resulting protein, such as not leading do an amendment of its amino acid sequence, or, if it leads to an amendment of the amino acid sequence, an amino acid is exchanged by a similar one, whereby the exchange does not affect the protein's function. For example, a silent mutation may be a point mutation by substitution of one nucleotide by another.

In a further preferred embodiment of the first and second aspect,
(i) the sequence of KoRVA Env is selected from (a) SEQ ID No: 2, (b) SEQ ID No: 6, wherein SEQ ID No: 6 is optionally C-terminally truncated for one or more of amino acids 645 to 659 of SEQ No: No: 6, and (c) a sequence of (a) or (b) lacking the signal peptide;
(ii) the sequence of the KoRVB Env is selected from (a) SEQ ID No: 4, (b) SEQ ID No: 8, wherein SEQ ID No: 8 is optionally C-terminally truncated for one or more of amino acids 652 to 666 of SEQ No: No: 8, and (c) a sequence of (a) or (b) lacking the signal peptide;
(iii) the sequence of the at least one nucleic acid encoding at least one KoRV Env comprises or consists of one or more of SEQ ID No: 1, SEQ ID No: 3, SEQ ID No: 5, wherein SEQ ID No: 5 is optionally truncated at the 3′ end for one or more of the 48 3′ terminal nucleotides, SEQ ID No: 7, wherein SEQ ID No: 7 is optionally truncated at the 3′ end for one or more of the 48 3′ terminal nucleotides, or a variant thereof comprising one or more silent mutations.

The nucleic acid encoding at least one KoRV Env may further be operably linked to (i) a heterologous promoter and/or (ii) a constitutive promoter and/or (iii) a poly A tail sequence.

The term “heterologous promoter”, as used herein, means any promoter that is not normally associated with a polynucleotide sequence in its natural environment. Suitable heterologous promoters are well-known to the person skilled in the art. For example, viral simian virus 40 (SV40) (e.g., early or late), cytomegalovirus (CMV) (e.g., immediate early), Moloney murine leukemia virus (MoMLV), Rous sarcoma virus (RSV), or herpes simplex virus (HSV) (thymidine kinase) promoters may be used.

The term “constitutive promoter”, as used herein, means an unregulated promoter that allows for continual transcription of its associated gene. Suitable constitutive promoters are well known to the person skilled in the art. For example, Human elongation factor-1 alpha (EF-1 alpha), simian virus 40 (SV40) early promoter, mouse mammary tumor virus (MMTV), human immunodeficiency virus (HIV) long terminal repeat (LTR) promoter, MoMLV promoter, an avian leukemia virus promoter, an Epstein-Barr virus immediate early promoter, a Rous sarcoma virus promoter, as well as human gene promoters such as, the actin promoter, the myosin promoter, the hemoglobin promoter, or the creatine kinase promoter may be used.

The term “poly A Tail sequence”, as used herein, means the addition of two or more adenosine monophosphates. In general, the poly A Tail sequence is located at the 3′ end of the nucleic acid.

In an also preferred embodiment of the first and second aspect, the nucleic acid encoding at least one KoRV Env is operably linked to (i) a heterologous promoter and/or (ii) a constitutive promoter and/or (iii) a poly A Tail sequence.

In a third aspect, the present invention relates to a pseudotyped viral vector particle which is pseudotyped with at least one KoRV Env.

All features specified above for the expression vector, expression cassette or pseudotyped viral vector particle of the first aspect of the invention as well as all features specified above for the nucleic acid of the second aspect of the invention are considered to also relate to the pseudotyped viral vector particle of the third aspect of the invention.

The pseudotyped viral vector particle according to the third aspect of the invention is pseudotyped with at least one KoRV Env. Preferably, the pseudotyped viral vector particle according to the third aspect of the invention is pseudotyped with one KoRV Env protein, for example with a KoRVA Env protein. Alternatively, the pseudotyped viral vector particle according to the third aspect of the invention may further be pseudotyped with at least two, three, four, five or more KoRV Env. These KoRV Env may for example differ in that they originate from different envelope glycoprotein variants, such as KoRVA and KoRVB, they may be chimeras of such variants as well as simply differ in being mutational variants of the same envelope glycoprotein variant.

Suitable viral vector particles for preparing pseudotyped viral vector particle of the first and third aspect are well-known to the person skilled in the art. For example, the viral vector particles for the pseudotyped viral vector particle of the first and third aspect may be selected from any virus species, such as retroviruses, adenoviruses, adeno-associated viruses, rhabdoviruses (such as vesicular stomatitis virus (VSV) or cocal virus), paramyxoviruses (such as measles virus, Nipah virus) and plant viruses and may also be hybrids of any virus species, such as those mentioned.

Preferably, the viral vector particles of the pseudotyped viral vector particle of the first and third aspect may be selected from retrovirus particles. A suitable retroviral vector particle may be selected from the group consisting of an oncoviral vector particle, including murine leukemia virus (MLV), avian leukosis virus (ALV), respiratory syncytial virus (RSV) or Mason-Pfizer monkey virus (MPMV) vector particles, a lentiviral vector particle, such as Human Immunodeficiency Virus (HIV), e.g. HIV-1 or HIV-2, Simian Immunodeficiency Virus (SIV), feline immunodeficiency virus (FIV), equine infectious anemia virus (EIAV) and caprine arthritis encephalitis virus (CAEV) vector particles, a gammaretrovirus particle and a spumaviral vector particle such as human foamy virus (HFV) vector particle.

More preferably, the viral vector particles may be selected from lentivirus vector particles and gammaretrovirus vector particles.

Retroviridae is a virus family with a single-stranded, diploid, positive-sense RNA genome that is reverse-transcribed into a DNA intermediate that is then incorporated into the host cell genome. Retroviridae-derived viruses are enveloped particles with a diameter of 80-120 nm. In the following, it is also referred to retroviridae as retroviruses.

Lentivirus is a genus of Retroviridae that cause chronic and deadly diseases characterized by long incubation periods, in the human and other mammalian species. The best-known lentivirus is the Human Immunodeficiency Virus HIV which can efficiently infect non-dividing cells, so lentiviral derived retroviral vectors are one of the most efficient methods of gene delivery.

Gammaretroviridae or is a genus of the Retroviridae family. Representative species are the murine leukemia virus and the feline leukemia virus. In the following, it is also referred to Gammaretroviridae as gammaretroviruses.

Suitable lentiviral vectors are well known in the art. For example, lentiviral vectors derived from human immunodeficiency virus (HIV-1), HIV-2 simian immunodeficiency virus, non-primate lentiviruses, feline immunodeficiency virus, EIAV, CAEV and bovine immunodeficiency virus, etc., may be used.

Moreover, gammaretroviral vectors are well known in the art. For example, gammaretroviral vectors derived from MoMLV (Moloney Murine Leukemia Virus) or MSCV (Murine Stem Cell Virus) may be used.

In a preferred embodiment of the pseudotyped viral vector particle of the first and third aspect, the viral vector particle is selected from a lentiviral or gammaretroviral vector particle.

Retroviral vector particle, lentiviral vector particles, and gammaretroviral vector particles are replication-deficient viral vector particles that are derived from the corresponding virus family. They contain Gag and Pol proteins, a single-stranded RNA genome and are typically pseudotyped with heterologous envelope proteins derived from other viruses. Viral vector particles of the invention are pseudotyped with at least one KoRV Env. The RNA genome of said viral vectors do not contain any viral gene to produce viral progeny, but, preferably, psi elements and LTRs that are required for efficient packing and reverse transcription in DNA. The DNA intermediate may contain a payload nucleic acid encoding a heterologous sequence of interest under the control of a suitable promoter, for example, the CMV promoter and the heterologous sequence of interest is expressed upon integration of said DNA into the genome of the host cell. The process of entering the host cell, delivering the RNA genome, integration and expression of the heterologous sequence of interest is called transduction. The minimal requirements of a gammaretrovirus or lentivirus based viral vector has been well-described in the art.

In the examples, a lentiviral particle pseudotyped with KORV Env proteins was used successfully for efficient transduction of NK cells.

The pseudotyped viral vector particle of the invention further comprises a lumen comprising a nucleic acid. The nucleic acid preferably comprises a viral nucleic acid comprising one or more of, such as for example all of, the following nucleic acid sequences: 5′ LTR (e.g., comprising U5 and lacking a functional U3 domain), Psi packaging element (Psi), central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

The pseudotyped viral vector particle is preferably replication defective.

The pseudotyped viral vector particle of the first and third aspect may further comprise a nucleic acid comprising at least one payload nucleic acid encoding a heterologous sequence of interest.

The term “payload nucleic acid”, as used herein, means any nucleic acid encoding a heterologous protein of interest. Thereby, the term “heterologous protein”, as used herein, means any protein that is not normally associated with the viral vector particle used in the third aspect in its natural environment. Preferably, the term “heterologous protein”, as used herein, means that the protein is in addition not present in the mammalian cell to be transduced. In general, suitable payload nucleic acids are well-known to the person skilled in the art. For example, the payload nucleic acid may encode a chimeric antigen receptor (CAR) comprising an antigen binding domain or comprise or constitute of a nucleic acid encoding a chimeric antigen receptor (CAR) comprising an antigen binding domain.

The nucleic acid comprising at least one payload nucleic acid may comprise further elements supporting translation and transduction of the heterologous protein of interest. The nucleic acid comprising at least one payload nucleic may be referred to as “transfer vector”. For example, the payload nucleic acid may be operably linked to a promoter, such as a constitutive or inducible promoter. For example, a CMV promoter may be used.

All features specified above for the nucleic acid of the first or third aspect of the invention are considered to also relate to the nucleic acid as comprised in the pseudotyped viral vector particle of the third aspect of the invention.

In an also preferred embodiment of the pseudotyped viral vector particle of the first and third aspect, the pseudotyped viral vector particle further comprises a nucleic acid comprising at least one payload nucleic acid encoding a heterologous sequence of interest.

In a fourth aspect, the present invention relates to a mammalian packaging cell line producing the pseudotyped viral vector particle according to the first or third aspect of the invention.

The term “mammalian packaging cell line”, as used herein, means any mammalian cell line that is generally capable of producing viral vector particles, e.g. by stably or transiently expressing viral structural proteins and replication enzymes (e.g., gag, pol and env) which can package viral vector particles. Suitable mammalian packaging cell lines are well known to the person skilled in the art and may be a cell line derived from any mammal, such as a human cell line, a murine cell line (such as a mouse cell line or a rat cell line), a hamster cell line, a canine cell line or a monkey cell line. Preferably, the mammalian packaging cell line is a human cell line.

Moreover, suitable mammalian packaging cell lines may be derived from various organs, such as kidney, ovary, placenta, bone marrow, lung, conjunctive tissue (such as fibroblast), liver, cervix, colon, brain or spleen. Suitable mammalian packaging cell lines may further be derived from cells of the immune system, such as macrophages. Also, neuronal cells or epithelial cells may be suitable. Further, suitable mammalian packaging cell lines may be derived from a healthy organism or an organism suffering from a disease. For example, suitable mammalian packaging cell lines may be derived from a tumor. Finally, suitable mammalian packaging cell lines may also be derived from embryonic or stem cells.

Examples of suitable mammalian cell lines are CHO cells, BHK cells, MDCK cells, C3H 10T1/2 cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, HEK 293 cells, HEK 293T cells, B-50 cells, 3T3 cells, NIH3T3 cells, HepG2 cells, Saos-2 cells, Huh7 cells, Hela cells, W163 cells, 211 cells, and 211A cells. Preferably, the mammalian packaging cell line are HEK 293T cells.

All features specified above for the pseudotyped viral vector particle of the first or third aspect of the invention are considered to also relate to the pseudotyped viral vector particle as comprised in the mammalian packaging cell line of the fourth aspect of the invention.

The mammalian packaging cell line according to the fourth aspect of the invention may further comprise the expression vector or the expression cassette according to the first aspect of the invention, the pseudotyped viral vector particle according to the first or third aspect of the invention, or the nucleic acid according to the second aspect of the invention. All features specified above for the expression vector or the expression cassette of the first aspect, the pseudotyped viral vector particle of the first or third aspect or the nucleic acid according to the second aspect are considered to also relate to the mammalian packaging cell line comprising such expression vector, expression cassette, pseudotyped viral vector particle or nucleic acid.

The mammalian packaging cell line according to the fourth aspect of the invention may comprise a nucleic acid comprising a long terminal repeat (LTR).

Further, the pseudotyped viral vector particle of the first and third aspect may comprise a nucleic acid comprising a long terminal repeat (LTR).

The term “long terminal repeat”, as used herein, means repeating nucleic acid sequences having e.g. 200-3000 base pairs, which usually flank genes and allow their transposition into a genome. Suitable LTRs are well-known to the person skilled in the art and may, e.g. be derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Suitable LTRs may e.g. be a 5′ LTR and/or a 3′ LTR. Suitable 5′ LTR and 3′LTR are well-known to the person skilled in the art and may, e.g. comprise U5 and lack a functional U3 domain.

The mammalian packaging cell line according to the fourth aspect of the invention may also comprise a nucleic acid comprising a Psi packaging element (Psi), which may e.g. be derived from Human immunodeficiency virus (HIV) or Simian immunodeficiency virus (SIV). Further, the pseudotyped viral vector particle of the first and third aspect may comprise a nucleic acid comprising a Psi packaging element (Psi), which may e.g. be derived from Human immunodeficiency virus (HIV) or Simian immunodeficiency virus (SIV).

The mammalian packaging cell line according to the fourth aspect of the invention may also comprise a nucleic acid comprising a central polypurine tract (cPPT)/central termination sequence (CTS) (e.g. DNA-flap). Further, the pseudotyped viral vector particle of the first and third aspect may comprise a nucleic acid comprising a central polypurine tract (cPPT)/central termination sequence (CTS) (e.g. DNA-flap). In general, the cPPT or the CTS comprise a sequence of at least nine nucleic acids, which each are either adenosine or guanosine nucleotides. Usually, the cPPT is located downstream of an encoded gene of interest (e.g. the heterologous protein) and upstream of the position, where a 3′ LTR may be located. The CTS may be located in the center of a viral genome in the integrase open reading frame. The nucleic acid in the mammalian packaging cell line according to the fourth aspect of the invention or in the pseudotyped viral vector particle of the first and third aspect may comprise a nucleic acid comprising a central polypurine tract (cPPT) or a central termination sequence (CTS) or both. Suitable nucleotide sequences for cPPT or CTS are well-known to the person skilled in the art. For example, the cPPT and the CTS may be chosen dependent on their capability of forming a DNA-flap between them.

The mammalian packaging cell line according to the fourth aspect of the invention may also comprise a nucleic acid comprising a Poly A tail sequence. Further, the pseudotyped viral vector particle of the first and third aspect may comprise a nucleic acid comprising a Poly A tail sequence. All features specified above for the Poly A tail sequence that may be linked to a nucleic acid in the expression vector, expression cassette or pseudotyped viral vector particle according to the first aspect or the nucleic acid according to the second aspect are considered to also relate to the Poly A tail sequence as comprised in the nucleic acid as comprised in the mammalian packaging cell line of the fourth aspect of the invention.

The mammalian packaging cell line according to the fourth aspect of the invention may also comprise a nucleic acid comprising a posttranscriptional regulatory element. Further, the pseudotyped viral vector particle of the first and third aspect may comprise a posttranscriptional regulatory element. The term “posttranscriptional regulatory element”, as used herein, means a nucleic acid sequence (e.g. a DNA sequence), that usually creates a tertiary structure, when it is transcribed, allowing the enhancement of intron-less heterologous gene expression. Suitable posttranscriptional regulatory elements are well-known to the person skilled in the art and may be derived from, e.g. Woodchuck Hepatitis virus (WPRE) or Hepatitis B virus (HPRE).

The mammalian packaging cell line according to the fourth aspect of the invention may also comprise a nucleic acid comprising a Rev response element (RRE), which may e.g. be derived from human immunodeficiency virus. Further, the pseudotyped viral vector particle of the first and third aspect may comprise a nucleic acid comprising a Rev response element (RRE), which may e.g. be derived from human immunodeficiency virus. Usually, the RRE is located in the Env coding region of unspliced and partially spliced viral mRNAs.

The mammalian packaging cell line according to the fourth aspect of the invention may comprise one or more of the following nucleic acid sequences: 5′ LTR, Psi packaging element (Psi), central polypurine tract (cPPT)/central termination sequence (CTS), Poly A tail sequence, a posttranscriptional regulatory element, a Rev response element (RRE), and 3′ LTR. Further, the pseudotyped viral vector particle of the first and third aspect may comprise one or more of the following nucleic acid sequences: 5′ LTR, Psi packaging element (Psi), central polypurine tract (cPPT)/central termination sequence (CTS), Poly A tail sequence, a posttranscriptional regulatory element, a Rev response element (RRE), and 3′ LTR.

The mammalian packaging cell line according to the fourth aspect of the invention may also comprise nucleic acid(s) encoding at least one nucleic acid encoding at least one Koala Retrovirus (KoRV) Envelope glycoprotein (Env). For example, the mammalian packaging cell line according to the fourth aspect of the invention may comprise one, two, three, four, or more nucleic acid(s) encoding at least one, two, three, four, or more nucleic acid(s) encoding at least one, two, three, four, or more Koala Retrovirus (KoRV) Envelope glycoprotein(s) (Env). All features specified above for the nucleic acid as used in the first or third aspect of the invention or according to the second aspect of the invention are considered to also relate to the nucleic acid as comprised in the mammalian packaging cell line of the fourth aspect of the invention.

The mammalian packaging cell line according to the fourth aspect of the invention may further comprise nucleic acid(s) encoding viral packaging protein(s). Suitable viral packaging proteins are well-known to the person skilled in the art and may be selected from any virus species, preferably from retroviruses, such as lentivirus. Examples for suitable viral packaging proteins are Gag, Pol, Rev and Tat. Preferably, the mammalian packaging cell line comprises according to the fourth aspect comprises nucleic acid(s) encoding viral packaging protein(s) selected from one or more of Gag, Pol, and Rev, and optionally Tat.

Methods for producing such mammalian packaging cell lines are well-known to the person skilled in the art and comprise, e.g. common transfection methods and may be supported by using transfection reagents, such as TransIT VirusGen (Mirus Bio), as exemplarily described in the examples below.

In a preferred embodiment of the mammalian packaging cell line of the fourth aspect of the invention, the mammalian packaging cell line comprises:
(i) one or more of the following nucleic acid sequences: 5′ LTR, Psi packaging element (Psi), central polypurine tract (cPPT)/central termination sequence (CTS), Poly A tail sequence, a posttranscriptional regulatory element, a Rev response element (RRE), and 3′ LTR;
(ii) nucleic acid(s) encoding at least one Koala Retrovirus (KoRV) Envelope glycoprotein (Env); and/or
(iii) nucleic acid(s) encoding viral packaging protein(s) selected from one or more of Gag, Pol, and Rev, and optionally Tat.

In a fifth aspect, the present invention relates to an in vitro method for delivery of at least one payload nucleic acid to at least one mammalian cell, comprising the steps:
(a) providing at least one mammalian cell,
(b) providing at least one pseudotyped viral vector particle according to the third aspect,
(c) contacting the at least one mammalian cell of (a) in vitro with the at least one pseudotyped viral vector particle of (b),
thereby obtaining at least one transduced mammalian cell comprising the at least one payload nucleic acid.

The pseudotyped viral vector particle in step (b) comprises a nucleic acid comprising at least one payload nucleic acid encoding a heterologous sequence of interest.

Preferably, the pseudotyped viral vector particle is selected from a lentiviral or gammaretroviral vector particle.

The in vitro method in the fifth aspect of the present invention relates to the delivery of at least one payload nucleic acid to at least one mammalian cell.

All features specified above for the payload nucleic acid in the context of the first and third aspect of the invention are considered to also relate to the payload nucleic acid as used in the in vitro method of the fifth aspect of the invention. The delivery of such at least one payload nucleic acid may for example mean the introduction of such payload nucleic acid into the mammalian cell.

The in vitro method for delivery of at least one payload nucleic acid to at least one mammalian cell according to the fifth aspect comprises at least three steps (a), (b) and (c).

### Step (a)

Step (a) of the method according to the fifth aspect is a step of providing at least one mammalian cell. If the at least one mammalian cell are at least two mammalian cells, these may be identical or mixtures of various mammalian cells. Mammalian cells may be provided in various reaction vessels, such as cell culture dishes, which are well-known to the person skilled in the art.

### Step (b)

Step (b) of the method according to the fifth aspect is a step of providing at least one pseudotyped viral vector particle according to the third aspect. This may also comprise providing two, three, four or more different pseudotyped viral vector particles according to the third aspect. All features specified above for the pseudotyped viral vector particle of the first and third aspect of the invention are considered to also relate to the pseudotyped viral vector particle as used in the in vitro method of the fifth aspect of the invention.

Step (c) Step (c) of the method according to the fifth aspect comprises contacting the at least one mammalian cell of (a) in vitro with the at least one pseudotyped viral vector particle of (b). In vitro methods of contacting mammalian cells and pseudotyped viral vector particles are well-known to the person skilled in the art. For example, the at least one pseudotyped viral vector particles may be added to the reaction vessel comprising the at least one mammalian cell as described for step (a).

In the in vitro method for delivery of at least one payload nucleic acid to at least one mammalian cell according to the fifth aspect comprising the at least three steps (a), (b) and (c), at least one transduced mammalian cell comprising the at least one payload nucleic acid is obtained.

Suitable transduction methods for viral vector particles are well-known to the person skilled in the art and, e.g. described in the examples below. For example, transduction may further be improved by using transduction enhancers, such as cationic polymers, lipids or peptides. For example, Vectofusin-1, a histidine-rich cationic amphipathic short peptide may be used, which enhances transduction with certain pseudotyped LVs, such as BaEV and GALV.

Suitable mammalian cells targetable by the in vitro method of the fifth aspect are in general all mammalian cell, such as human cells, murine cells (such as mouse cells or rat cells), hamster cells, canine cells or monkey cells.

Preferably, the at least one mammalian cell is a human cell.

Suitable mammalian cells the payload nucleic acid may be delivered to may be cells of various organs, such as kidney, ovary, placenta, bone marrow, lung, conjunctive tissue (such as fibroblasts), liver, cervix, colon, brain or spleen. Suitable mammalian cells the payload nucleic acid may be delivered to may be cells of the immune system, such as macrophages. Further, suitable mammalian cells the payload nucleic acid may be delivered to may be cells of various tumors. Further, suitable mammalian cells the payload nucleic acid may be delivered to may be cells which are non-tumor cells and/or non-hyperproliferative cells. Suitable mammalian cells the payload nucleic acid may be delivered to may also be embryonic cells. In one embodiment, the mammalian cells are not human embryonic cells. Suitable mammalian cells the payload nucleic acid may be delivered to may further be hematopoietic cells, iPS (induced pluripotent stem) cells, stem cells, or immune cells (e.g. T cells, NK cells, B cells, dendritic cells, monocytes or macrophages). Preferably, the at least one mammalian cell is selected from a hematopoietic cell, an iPS cell, a stem cell, or an immune cell, optionally wherein the immune cell is selected from a T cell, an NK cell, a B cell, a dendritic cell, a monocyte, a macrophage, or a mixture thereof. More preferably, the at least one mammalian cell is selected from a T cell or an NK cell. Even more preferably, the at least one mammalian cell is a T cell.

In Example 1, it was found that the envelope proteins KoRVA and KoRVB as well as their combination designated KORVAB enabled highly efficient transduction of NK cells, which was even better than previously described viral envelopes including BaEV on the day of isolation of NK cells; i.e. for NK cells which were not previously activated. Similar to the results of Example 1, the data of Example 2 confirm that the transduction efficiency in fresh, primary NK cells of each of KoRVA, KoRVB or KORVAB enveloped lentiviral vector particles is superior in comparison to BaEV pseudotyped lentiviral vector particles.

Further, peripheral blood mononuclear cells (PBMCs) were successfully modified in Example 3 with KoRV-A- or KoRV-B-enveloped lentiviruses. PBMC samples therefore represent an example of a mixture of mammalian cells. Further, it was found that all cell types in PBMCs, B cells (CD19+ B cells in the example), monocytes (CD14+ monocytes in the example), NK cells (CD56+/CD3− NK cells in the example) and T cells (CD3+ T cells in the example) can be modified using KoRVA- and/or KoRVB-enveloped lentiviruses. In particular, monocytes and B cells were found to be more susceptible to KoRVA- or KoRVB-mediated gene transfer than NK cells and T cells.

The at least one mammalian cell may be activated prior to step (c) or may not be activated prior to step (c). Suitable methods for activating a mammalian cell are well-known to the person skilled in the art. For example, mammalian cells, especially, immune cells, such as T cells, NK cells, dendritic cells, monocytes or macrophages, may be activated. Suitable agents for activating immune cells are well-known to the person skilled in the art and usually depend on the cell type. For example, interleukins may be used to activate NK cells, such as IL-2 and/or IL-15. Macrophages may for example be activated using lipopolysaccharides and interferons (IFN), such as IFN-gamma. T cells may for example be activated using antigen-presenting cells (APCs), anti-CD3 and/or anti-CD28 antibodies. Preferably, the at least one mammalian cell is activated prior to step (c) or is not activated prior to step (c). More preferably, the T cell, NK cell, dendritic cell, monocyte, macrophage, or mixture thereof, is not activated prior to step (c).

In Examples 1 and 2, a superior transduction of NK cells was found for KoRVA and KoRVB Env for NK cells which were not activated.

The term “activation” as used herein refers to inducing physiological changes with a cell that increase target cell function, proliferation and/or differentiation.

Activation of mammalian immune cells, in particular NK cells, can be achieved e.g. as described in WO2019/121945. In particular, activation of NK cells can be achieved by addition of at least one cytokine or feeder cells or membrane particles of feeder cells or a with an NK cell activation reagent to said NK cells. Said at least one cytokine may be IL-2 and/or IL-15, or a combination of IL2 and/or IL-I 5 and an IL-1 family cytokine, wherein said IL-1 family cytokine is IL-18, IL-33 or IL-1beta.

The at least one payload nucleic acid may encode one or more protein(s) and/or RNA(s) of interest. Further, the at least one payload nucleic acid may encode two, three, four, five or more protein(s) and/or RNA(s) of interest.

The one or more protein(s) of interest encoded by the at least one payload nucleic acid may be any protein. For example, the one or more protein(s) of interest encoded by the at least one payload nucleic acid is selected from a Chimeric Antigen Receptor (CAR), a T cell receptor (TCR), a chemokine receptor, an NK cell receptor, an immunoregulatory protein, a cytokine, an antibody and/or a targeted endonuclease. Further, if two or more proteins of interest are encoded by the at least one payload nucleic acid, these may be selected from different variants (e.g. mutants) of the same protein or be a mixture of a Chimeric Antigen Receptor (CAR), a T cell receptor (TCR), a chemokine receptor, an NK cell receptor, an immunoregulatory protein, a cytokine, an antibody and/or a targeted endonuclease. Further, if two or more proteins of interest are encoded by the at least one payload nucleic acid, these may also be selected from two of these same types, such as two Chimeric Antigen Receptors (CAR), two T cell receptors (TCR), two chemokine receptors, two NK cell receptors, two immunoregulatory proteins, two cytokines, two antibodies and/or two targeted endonucleases.

The one or more RNA(s) of interest encoded by the at least one payload nucleic acid may be any RNA. For example, the one or more RNA (s encoded by the at least one payload nucleic acid) of interest is selected from a ribozyme, a gRNA, an antisense RNA, an siRNA a miRNA or combinations thereof.

Preferably, the at least one payload nucleic acid encodes one or more protein(s) and/or RNA(s) of interest.

Preferably, the one or more protein(s) of interest encoded by the at least one payload nucleic acid is selected from a Chimeric Antigen Receptor (CAR), a T cell receptor (TCR), a chemokine receptor, an NK cell receptor, an immunoregulatory protein, a cytokine, an antibody, a targeted endonuclease, and/or the one or more RNA(s) encoded by the at least one payload nucleic acid of interest is selected from a ribozyme, a gRNA, an antisense RNA, an siRNA a miRNA or combinations thereof.

The at least one payload nucleic acid may be integrated into the mammalian cell genome. Further, if two or more payload nucleic acids are used, these may be partly introduced into the mammalian cell genome and partly not introduced into the mammalian cell genome. Methods for integrating payload nucleic acid into the genome of a mammalian cell are well-known to the person skilled in the art. In particular, the pseudotyped viral vector particle of the invention may mediate, after transduction, the stable integration into the genome. Preferably, the at least one payload nucleic acid is integrated into the mammalian cell genome. Preferably, the at least one payload nucleic acid is stably integrated into the mammalian cell genome.

Further, all features specified above for the at least one KoRV Env of the first, second, third or fourth aspect of the invention are considered to also relate to the at least one KoRV Env as used in the in vitro method of the fifth aspect of the invention. Preferably, the at least one KORV Env used in the in vitro method of the fifth aspect is KORVA.

Further, all features specified above for the at least one viral vector particle of the first or third aspect of the invention are considered to also relate to the at least one viral vector particle as used in the in vitro method of the fifth aspect of the invention. Preferably, the at least one viral vector particle is a lentiviral vector particle.

Preferably,
(i) the at least one mammalian cell is a human cell and/or
(ii) the at least one mammalian cell is selected from a hematopoietic cell, an iPS cell, a stem cell, or an immune cell, optionally wherein the immune cell is selected from a T cell, an NK cell, a B cell, a dendritic cell, a monocyte, a macrophage, or a mixture thereof, and/or
(iii) the at least one payload nucleic acid encodes one or more protein(s) and/or RNA(s) of interest, and/or
(iv) the at least one mammalian cell is activated prior to step (c) or is not activated prior to step (c), and/or
(v) the at least one payload nucleic acid is integrated into the mammalian cell genome.

Optionally,
(a) the at least one mammalian cell is selected from a T cell, an NK cell, a B cell, a dendritic cell, a monocyte, a macrophage, or a mixture thereof, optionally wherein:
   (i) the T cell, NK cell, B cell, dendritic cell, monocyte, macrophage, or mixture thereof, is not activated prior to step (c), and/or
   (ii) the one or more protein(s) of interest encoded by the at least one payload nucleic acid is selected from a Chimeric Antigen Receptor (CAR), a T cell receptor (TCR), a chemokine receptor, an NK cell receptor, an immunoregulatory protein, a cytokine, an antibody, a targeted endonuclease, and/or the one or more RNA(s) encoded by the at least one payload nucleic acid of interest is selected from a ribozyme, a gRNA, an antisense RNA, an siRNA a miRNA or combinations thereof, and/or
   (iii) the at least one KoRV Env is KoRVA, and/or
(b) the at least one viral vector particle is a lentiviral vector particle.

In a preferred embodiment of the method of the fifth aspect:
(i) the at least one mammalian cell is a human cell and/or
(ii) the at least one mammalian cell is selected from a hematopoietic cell, an iPS cell, a stem cell, or an immune cell, optionally wherein the immune cell is selected from a T cell, an NK cell, a B cell, a dendritic cell, a monocyte, a macrophage, or a mixture thereof, and/or
(iii) the at least one payload nucleic acid encodes one or more protein(s) and/or RNA(s) of interest, and/or
(iv) the at least one mammalian cell is activated prior to step (c) or is not activated prior to step (c), and/or
(v) the at least one payload nucleic acid is integrated into the mammalian cell genome,
   optionally wherein:
(a) the at least one mammalian cell is selected from a T cell, an NK cell, a B cell, a dendritic cell, a monocyte, a macrophage, or a mixture thereof, optionally wherein:
   (i) the T cell, NK cell, B cell, dendritic cell, monocyte, macrophage, or mixture thereof, is not activated prior to step (c), and/or
   (ii) the one or more protein(s) of interest encoded by the at least one payload nucleic acid is selected from a Chimeric Antigen Receptor (CAR), a T cell receptor (TCR), a chemokine receptor, an NK cell receptor, an immunoregulatory protein, a cytokine, an antibody, a targeted endonuclease, and/or the one or more RNA(s) is selected from a ribozyme, a gRNA, an antisense RNA, an siRNA a miRNA or combinations thereof, and/or
   (iii) the at least one KoRV Env is KoRVA, and/or
(b) the at least one viral vector particle is a lentiviral vector particle.

In a sixth aspect, the present invention relates to a transduced mammalian cell obtainable by the method of the fifth aspect, optionally wherein the transduced mammalian cell is for use in adoptive cell therapy or transplantation.

All features specified above for the in vitro method of the fifth aspect of the invention are considered to also relate to the method as used in the sixth aspect of the invention.

The term “adoptive cell therapy”, as used herein, means any type of immunotherapy in which T cells are administrated to a patient to help the body fight diseases, such as cancer. For example, in cancer therapy, immune cells, such as NK cells, T cells or macrophages, or cells from the tumor may be isolated from the patient, cultivated and grown to larger numbers in the laboratory, subsequently modified and then be introduced into the patient. Amendments of the cultivated cells may e.g. be performed using pseudotyped viral vector particles as described in the first or third aspect of the invention, e.g. comprising the expression vector, the expression cassette or the nucleic acid according to the first and second aspect of the invention, respectively. Methods for amending the cells isolated from the patient are well-known to the person skilled in the art and are also exemplarily described in the fifth aspect of the invention or in the examples below. Examples of adoptive cell therapy are, e.g. chimeric antigen receptor T-cell (CAR T-cell) therapy or tumor-infiltrating lymphocyte (TIL) therapy. Adoptive cell therapy that uses immune cells, such as NK cells, T cells or macrophages from a donor may also be used in the treatment of some types of cancer and some infections. Accordingly, the adoptive cell therapy may use autologous, allogeneic or xenogeneic cells.

The term “transplantation” or “grafting”, as used herein, means any removal of organ, tissue or cells from the body of a donor to the body of a recipient in order to replace a lacking, damaged or malfunctioning organ, tissue or cells in the recipient's body.

In a seventh aspect, the present invention relates to the in vitro use of at least one KORV Env glycoprotein, or of at least one nucleic acid encoding a KoRV Env glycoprotein, or of a mammalian packaging cell line of the fourth aspect of the invention, or of an expression vector or expression cassette according to the first aspect of the invention, or of a pseudotyped viral vector particle according to the first or third aspect of the invention, or of a nucleic acid of the second aspect of the invention, for:
(i) delivery of at least one payload nucleic acid encoding one or more protein(s) and/or RNA(s) of interest to a mammalian cell;
(ii) stably transducing a mammalian cell; and/or
(iii) transducing mammalian immune cells without pre-activation of the immune cells.

For example, the method can be used to modify and genetically engineer mammalian cells of interest by stably integrating at least one heterologous sequence of interest. The transduced mammalian cells obtained thereby may be used for research purposes, such as for screening purposes. Alternatively, the transduced mammalian cells obtained thereby may be used for therapeutic or diagnostic purposes, e.g. for adoptive cell therapy or transplantation, as described above. The transduced mammalian cells may cultivated and thereby amplified, and optionally stored until further use.

All features specified above for the at least one KoRV Env glycoprotein, or for the at least one nucleic acid encoding a KoRV Env glycoprotein, or for a mammalian packaging cell line of the fourth aspect of the invention, or for an expression vector or expression cassette according to the first aspect of the invention, or for a pseudotyped viral vector particle according to the first or third aspect of the invention, or for a nucleic acid of the second aspect of the invention are considered to also relate to the in vitro use in the seventh aspect of the invention.

The in vitro use of at least one KoRV Env glycoprotein, or of at least one nucleic acid encoding a KoRV Env glycoprotein, or of a mammalian packaging cell line of the fourth aspect of the invention, or of an expression vector or expression cassette according to the first aspect of the invention, or of a pseudotyped viral vector particle according to the first or third aspect of the invention, or of a nucleic acid of the second aspect of the invention may be for
(i) delivery of at least one payload nucleic acid encoding one or more protein(s) and/or RNA(s) of interest to a mammalian cell;
(ii) stably transducing a mammalian cell; and/or
(iii) transducing mammalian immune cells without pre-activation of the immune cells.

All features specified above for the delivery of at least one payload nucleic acid encoding one or more protein(s) and/or RNA(s) of interest to a mammalian cell in the fifth aspect are considered to also relate to the in vitro use in the seventh aspect of the invention.

Further, a transduction of a mammalian cell may be considered stably transduced, if the at least one payload nucleic acid is maintained in the genome of the mammalian cell for at least 5, 10, 15, 20 or 30 cell division cycles.

Preferably, such stable transduction is achieved without pre-activation of the immune cells. As explained above, it was surprisingly found that it is possible to efficiently transduce human NK cells using nucleic acids encoding a KoRV Env glycoproteins. Accordingly, in a preferred embodiment, the immune cells are transduced without pre-activation. In this context, all features specified above for pre-activation of immune cells as it may be applied in the method of the fifth aspect are considered to also relate to the in vitro use in the seventh aspect of the invention.

In this context, all features described above for the first, second or third aspect of the invention, where applicable, also apply to the fourth, fifth, sixth or seventh aspect of the invention, such as the features related to the expression vector, expression cassette, pseudotyped viral vector particle, KoRV Env or nucleic acid.

In general, the disclosure is not limited to the particular methodology, protocols, and reagents described herein because they may vary. Further, the terminology used herein is for the purpose of describing particular embodiments only and is not intended to limit the scope of the present disclosure. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the disclosure. Although any methods and materials similar or equivalent to those described herein can be used in the practice as presented herein, the specific methods, and materials are described herein.

The disclosure is further illustrated by the following figures and examples, although it will be understood that the figures and examples are included merely for purposes of illustration and are not intended to limit the scope of the disclosure unless otherwise specifically indicated.

### EXAMPLES

### General Methods

### KORV Pseudovirus Generation in HEK293T Cells and NK Cell Transduction

The viral vector particles are produced by viral vector systems of the so-called 2nd or 3rd generation in 293T cells. Vectors containing viral packaging elements, as well as a transfer plasmid containing the transgene, and the newly developed sequence described here, which contains either the modified envelope protein of KoRVA or KoRVB, are used for transfection (FIG. 2). A mixture of both vectors (KoRVA and KoRVB) can also be used successfully.

In detail, HEK293T cells were seeded 24-48 h prior to transfection to reach a density of 50-70% confluence at day of transfection, and grown in DMEM medium+10% FCS+1% Pen/Strep. At the day of transfection, a DNA mix was prepared with a lentiviral vector containing the payload nucleic acid (here designated as gene of interest (GOI)), and the plasmids encoding the viral packaging machinery, in the ratio 5:4:1:1 (GOI: gag/pol: rev: KoRV). For example, for the generation of 10 mL viral supernatants in a 100-mm tissue culture dish, these vectors can be 5 μg EF1a-mVenus-H2B-IRES-mCherry-PGK-Puro: 4 μg pMDLg-pRRE-gag/pol: 1 μg pRSV-Rev:1 μg KoRVA_pTwist_CMV_BetaGlobin_WPRE_Neo or KoRVB_pTwist_CMV_BetaGlobin_WPRE_Neo, or similar. When KoRVA and KoRVB were simultaneously used, they were mixed in a 1:1-ratio. The plasmid mixture was added to serum-free medium, mixed, and then a suitable transfection reagent was added, for example, TransIT VirusGen (Mirus Bio). It was gently pipetted to mix and incubated at room temperature for 15 minutes. The mixture was added drop-wise to different areas of the cell culture dish containing the HEK293T cells. The culture vessel was gently rocked back-and-forth and from side-to-side to evenly distribute the complexed DNA. It was incubated at 37° C. in 5% CO2 for 12-18 hours, the growth medium was exchanged, and it was incubated for another 48-60 hours. The virus-containing supernatants were collected into a fresh tube, centrifuged for 10 minutes, 300×g, 4° C. to remove cells and cellular debris and transferred to a new sterile vessel. The supernatants were immediately used or frozen in −80° C. for later use.

NK cells were prepared in 5 million per mL in NK MACS medium (Miltenyi). For example, 125.000 NK cells were used for a 48-well-plate, so they were resuspended in 25 μL per transduction and mixed well. 250 μL of the viral supernatant per well for each transduction was pipetted in a 48-well-plate. 1:100 Vectofusin (Miltenyi) transduction enhancer was added to the supernatants, then 25 μL of the NK cell suspensions were added to each well. Spinfection was performed at 400×g, 37° C. for 60 min. 250 μL NK MACS medium were added and put in 37° C. incubator for around 3 days, then transgene expression was analyzed using flow cytometry.

Other mammalian immune cells can be transduced in a similar manner.

Lentiviral vector EF1a-mVenus-H2B-IRES-mCherry-PGK-Puro used in the experiments contains two human promoters and encodes 3 payload nucleic acids, encoding two fluorescent markers, and 1 selection marker, respectively.

### KORV Env Sequences

KoRVA and KoRVB Env, two Env variants from the Koala retrovirus, were successfully identified as viral surface structures to enable successful and efficient gene transfer into NK cells. In addition, KoRVA and KoRVB Env were further modified successfully for enhanced functionality. To this end, the C-terminal R-peptide was removed, which is normally proteolytically cleaved during the viral life cycle in similar viruses to allow fusion activity of the viral vector particle with the target cell (cf. FIG. 1 showing the domain structure KoRVA Env and KoRVB Env). In the examples herein, a sequence encoding KoRVA Env wherein the C-terminal R-peptide is removed (SEQ ID No: 2) and/or a sequence encoding KoRVB Env wherein the C-terminal R-peptide is removed (SEQ ID No: 4) were used. In case a combination of KoRVA Env and KoRVB Env are used, designated herein as KORVAB, the sequences SEQ ID No: 2 and SEQ ID No: 4 were included and transfected on separate vectors. However, it is also possible to use a single vector comprising the KoRVA Env sequence and the KoRVB Env sequence. The sequences of SEQ ID No: 2 and 6 include the N-terminal signal peptide. In the pseudotyped viral vector particles generated herein, the signal peptide is removed from KoRVA Env and KoRVB Env, respectively. The pseudotyped viral vector particles generated comprise mature KoRVA Env or KoRVB Env, wherein, further, the C-terminal R-peptide is removed.

### BaEV-Sequences

BaEV Env sequences were used herein for comparison. The sequences of BaEV Env were used as previously published by Girard-Gagnepain et al. 2014. This BaEV envelope was also used in previous publications to transduce activated NK cells (Bari et al., 2019, and Colamartino et al., 2019; see also WO2013/045639 A1 and WO2019/121945 A1). The BaEV Env sequences used herein for comparison also lacks the C-terminal R-peptide.

### Example 1-Comparison of Transduction Efficiency of KoRVA, KoRVB, KORVAB and BaEV Enveloped Pseudoviruses in Peripheral Blood NK Cells

KoRV pseudovirus generation in HEK293T cells and NK cell transduction was performed as described above. For each envelope protein, KoRVA, KoRVB, a mixture thereof and BaEV, the transduction efficiency was investigated for three different donors.

In detail, isolated NK cells were transduced on the day of isolation, alternatively on day 3, 7 and 21 after activation in NK MACS medium (Miltenyi) containing 500 U/mL IL-2 and 140 U/mL IL-15 (Peprotech), with KoRV- or BaEV-pseudotyped lentiviral vector particles. Transduction efficiency was determined 72 hours after transduction by flow cytometric analysis of the fluorescent protein mVenus.

It was found that the envelope proteins KoRVA and KoRVB as well as their combination designated KoRVAB enabled the efficient production of pseudotyped viral vector particles containing a transfer gene of interest, i.e. a payload nucleic acid, and are therefore suitable for transduction. Moreover, it was surprisingly found that the transduction of NK cells is highly efficient and was even better than previously described viral envelopes, including BaEV on the day of isolation of NK cells; i.e. for NK cells which were not previously activated. In particular, it was found that a combination of KoRVA and KoRVB was superior in the transduction of non-activated NK cells on the day of isolation (FIG. 3). Accordingly, a superior transduction of NK cells was found for KoRVA and KoRVB Env for NK cells which were not activated.

### Example 2-Comparison of Transduction Efficiency of KoRVA, KoRVB, KORVAB and BaEV Enveloped Pseudoviruses in Fresh, Primary NK Cells

KoRV pseudovirus generation in HEK293T cells and NK cell transduction was performed as described above. For each envelope protein KoRVA, KoRVB, a mixture thereof and BaEV, the transduction efficiency was investigated for three different donors.

In detail, NK cells isolated from peripheral blood were transduced with KoRV- or BaEV-pseudotyped lentiviral vector particles on the day of isolation. Transduction efficiency was determined 72 hours after transduction by flow cytometric analysis of the fluorescent protein mVenus. The transduction efficiency averaged over three donors and the standard deviation are plotted. Statistical analysis was performed using one-way analysis of variance (ANOVA), *=p<0.05.

Similar to the results of Example 1, the data of Example 2 confirm that the transduction efficiency in fresh, primary NK cells of each of KoRVA, KoRVB or KORVAB enveloped lentiviral vector particles is superior in comparison to BaEV pseudotyped lentiviral vector particles. A mixture of KoRVA and KoRVB for preparing pseudotyped lentiviral vector particles for subsequent transduction even resulted nearly a duplication of transduction efficiency as compared to BaEV Env pseudotyped lentiviral vector particles. Similar advantageous results were obtained with KoRVA Env pseudotyped lentiviral vector particles.

### Example 3: Transduction of Peripheral Blood Mononuclear Cells (PBMCs) with KoRV-A- or KoRV-B-Enveloped Lentiviruses

PBMCs from peripheral blood of 6 healthy donors were isolated and immediately transduced (without separating the immune cell populations). Using the fluorescent reporter gene mVenus, transduction efficiencies were determined in CD19+B cells, CD14+ monocytes, CD56+/CD3-NK cells and CD3+ T cells 3 days after transduction.

It was found that all cell types can be modified using KoRVA- and/or KoRVB-enveloped lentiviruses. In particular, monocytes and B cells were found to be more susceptible to KoRVA- or KoRVB-mediated gene transfer than NK cells and T cells.

### REFERENCES

Girard-Gagnepain A. et al. (“Baboon envelope pseudotyped LVs outperform VSV-G-LVs for gene transfer into early-cytokine-stimulated and resting HSCs”), Blood (2014) 124 (8): 1221-1231; doi: 10.1182/blood-2014-02-558163.
Bari R. et al. (“A Distinct Subset of Highly Proliferative and Lentiviral Vector (LV)-Transducible NK Cells Define a Readily Engineered Subset for Adoptive Cellular Therapy”), Frontiers in Immunology, 2019, 10:2001. doi: 10.3389/fimmu.2019.02001. Erratum in: Front Immunol. 2019 Dec. 4; 10:2784.
Colamartino A. B. L. et al., (“Efficient and Robust NK-Cell Transduction With Baboon Envelope Pseudotyped Lentivector”), Frontiers in Immunology, 2019, 10:2873. doi: 10.3389/fimmu.2019.02873 . . . .
WO2013/045639
WO2019/121945
WO2021/041322
WO2021/127076

1. An expression vector, an expression cassette or a pseudotyped viral vector particle comprising at least one nucleic acid encoding at least one Koala Retrovirus (KoRV) Envelope glycoprotein (Env).

2. The expression vector, expression cassette or pseudotyped viral vector particle according to claim 1, wherein the at least one nucleic acid encodes at least one KORV Env which lacks a C-terminal R-peptide.

3. A nucleic acid encoding at least one KoRV Env which lacks a fusion inhibitory R-peptide (R-peptide) or lacks part of the R-peptide.

4. The expression vector, expression cassette or pseudotyped viral vector particle according to claim 1, wherein the at least one KoRV Env is selected from KoRVA, KoRVB and a combination thereof.

5. The expression vector, expression cassette or pseudotyped viral vector particle according to claim 4, wherein:
(i) the sequence of KoRVA Env is selected from (a) SEQ ID No: 2, (b) SEQ ID No: 6, wherein SEQ ID No: 6 is optionally C-terminally truncated for one or more of amino acids 645 to 659 of SEQ No: No: 6, and (c) a sequence of (a) or (b) lacking the signal peptide;
(ii) the sequence of the KoRVB Env is selected from (a) SEQ ID No: 4, (b) SEQ ID No: 8, wherein SEQ ID No: 8 is optionally C-terminally truncated for one or more of amino acids 652 to 666 of SEQ No: No: 8, and (c) a sequence of (a) or (b) lacking the signal peptide; and/or
(iii) the sequence of the at least one nucleic acid encoding at least one KORV Env comprises or consists of one or more of SEQ ID No: 1, SEQ ID No: 3, SEQ ID No: 5, wherein SEQ ID No: 5 is optionally truncated at the 3′ end for one or more of the 48 3′ terminal nucleotides, SEQ ID No: 7, wherein SEQ ID No: 7 is optionally truncated at the 3′ end for one or more of the 48 3′ terminal nucleotides, or a variant thereof comprising one or more silent mutations.

6. The expression vector, expression cassette or pseudotyped viral vector particle according to claim 1, to 5, wherein the nucleic acid encoding at least one KoRV Env is operably linked to (i) a heterologous promoter and/or (ii) a constitutive promoter and/or (iii) a poly A Tail sequence.

7. A pseudotyped viral vector particle which is pseudotyped with at least one KoRV Env.

8. The pseudotyped viral vector particle according to claim 1, wherein the viral vector particle is selected from a lentiviral or gammaretroviral vector particle.

9. The pseudotyped viral vector particle according to claim 1 further comprising a nucleic acid comprising at least one payload nucleic acid encoding a heterologous sequence of interest.

10. A mammalian packaging cell line producing the pseudotyped viral vector particle according to claim 1.

11. The mammalian packaging cell line of claim 10, wherein the mammalian packaging cell line comprises:
(i) one or more of the following nucleic acid sequences: 5′ LTR, Psi packaging element (Psi), central polypurine tract (cPPT)/central termination sequence (CTS), Poly A tail sequence, a posttranscriptional regulatory element, a Rev response element (RRE), and 3′ LTR;
(ii) nucleic acid(s) at least one Koala Retrovirus (KoRV) Envelope glycoprotein (Env); and/or
(iii) nucleic acid(s) encoding viral packaging protein(s) selected from one or more of Gag, Pol, and Rev, and optionally Tat.

12. A method for in vitro delivery of at least one payload nucleic acid to at least one mammalian cell, comprising the steps:
(a) providing at least one mammalian cell,
(b) providing at least one pseudotyped viral vector particle according to claim 9, and
(c) contacting the at least one mammalian cell of (a) in vitro with the at least one pseudotyped viral vector particle of (b), thereby obtaining at least one transduced mammalian cell comprising the at least one payload nucleic acid.

13. The method of claim 12, wherein:
(i) the at least one mammalian cell is a human cell, and/or
(ii) the at least one mammalian cell is selected from a hematopoietic cell, an iPS cell, a stem cell, or an immune cell, optionally wherein the immune cell is selected from a T cell, an NK cell, a B cell, a dendritic cell, a monocyte, a macrophage, or a mixture thereof,
(iii) the at least one payload nucleic acid encodes one or more protein(s) and/or RNA(s) of interest,
(iv) the at least one mammalian cell is activated prior to step (c) or is not activated prior to step (c), and/or
(v) the at least one payload nucleic acid is integrated into the mammalian cell genome,
optionally wherein:
(a) the at least one mammalian cell is selected from a T cell, an NK cell, a B cell, a dendritic cell, a monocyte, a macrophage, or a mixture thereof,
optionally wherein:
(i) the T cell, NK cell, B cell, dendritic cell, monocyte, macrophage, or mixture thereof, is not activated prior to step (c),
(ii) the one or more protein(s) of interest encoded by the at least one payload nucleic acid is selected from a Chimeric Antigen Receptor (CAR), a T cell receptor (TCR), a chemokine receptor, an NK cell receptor, an immunoregulatory protein, a cytokine, an antibody, a targeted endonuclease, and/or the one or more RNA(s) encoded by the at least one payload nucleic acid of interest is selected from a ribozyme, a gRNA, an antisense RNA, an siRNA a miRNA or combinations thereof, and/or
(iii) the at least one KoRV Env is KoRVA, and/or
(b) the at least one viral vector particle is a lentiviral vector particle.

14. A transduced mammalian cell obtainable by the method of claim 12, optionally wherein the transduced mammalian cell is for use in adoptive cell therapy or transplantation.

15. A method of delivery of at least one payload nucleic acid encoding one or more protein(s) and/or RNA(s) of interest to a mammalian cell utilizing at least one KoRV Env glycoprotein, or of at least one nucleic acid encoding a KoRV Env glycoprotein, or of a mammalian packaging cell line of claim 10, for:

16. A method of stably transducing a mammalian cell utilizing at least one KoRV Env glycoprotein, or of at least one nucleic acid encoding a KoRV Env glycoprotein, or of a mammalian packaging cell line of claim 10.

17. A method of transducing mammalian immune cells without pre-activation of the immune cells utilizing at least one KoRV Env glycoprotein, or of at least one nucleic acid encoding a KoRV Env glycoprotein, or of a mammalian packaging cell line of claim 10.

18. A method of delivery of at least one payload nucleic acid encoding one or more protein(s) and/or RNA(s) of interest to a mammalian cell utilizing the expression vector, expression cassette, or pseudotyped viral vector particle according to claim 1.

19. A method of stably transducing a mammalian cell utilizing the expression vector, expression cassette, or pseudotyped viral vector particle according to claim 1.

20. A method of transducing mammalian immune cells without pre-activation of the immune cells utilizing the expression vector, expression cassette, or pseudotyped viral vector particle according to claim 1.


SEQUENCE LISTING (8 sequences)

SEQ ID NO: 1 - DNA, 1935 bp, synthetic construct
  misc_feature 1..1935: note = KorV-A sequence truncated for C-terminal R-peptide
  source 1..1935: mol_type = other DNA
atgcttctcatctcaaacccgcgccacctggggcacccgatgagtcctgggaactggaaaagactgatcatcctcttaagttgcgtgttcggcggcgccgaaatgaatcaacagcataacaaccctcaccaacccatgactctcacctggcaggtactgtcccagacgggaagtgtcgtttgggaaaagaaagcagtcgagccaccctggacgtggtggccctctcttgagcctgatgtgtgcgcgctggtagccggtcttgagtcctgggacatcccggaactcactgcatcggcctctcaacaggccaggccccctgattcaaactatgaacatgcttataatcagatcacttggggaaccctgggatgtagctatccccgggccagaacaagaattgctaggtcccagttctacgtgtgcccccgggatggccgttccctgtccgaggcccggaggtgcggaggacttgaatctttgtactgtaaagagtggggttgtgagaccgcgggaaccgcctactggcaaccccggtcctcgtgggaccttataactgtaggccagggtcaccctacagggacatgcgagcgcaccggttggtgcaacccactcaagatagagttcaccgagcctggcaaacggttccgaaactggctacaggggcgaacctggggtctaaggttctatgtgactggacacccaggcgtgcagttgaccatacgactagtgatcacaagtcccccaccggtagtagtgggccccgatcctgtcctcgcagaacaaggacctcccagaaaaattcccttcctccccagagtgccggttcctaccctatcacccccagcgtcccctatccccacggtacaggccagccccccggcccctagtactccatctcccaccacgggcgaccggctctttggcctcgtacaaggggcctttctggccttgaatgccaccaacccggaggccacggagtcttgctggctctgtctagccctcggccctccttattacgaaggaatcgcaactccagggcaagtcacttatgcctccacagattcccaatgtcgctggggaggaaaagggaagctcaccctcactgaagtctctggactcgggttatgtatagggaaagtacctcctactcaccaacatctctgcaacctgactatacccttaaacgcctcacatactcacaagtacctcctcccctccaaccgcagctggtgggcttgtaattctggcctcaccccctgcctctctacatcagtcttcaaccagtctaacgatttctgtatccagatccagcttgtccctcgcatctactatcacccagacggtaccttgctacaggcctatgagtccccccactcaagaaacaagagagagcctgtctcactcaccctggctgtccttctcggattaggggtcgcagcaggtataggtaccggctcgaccgccctaataaaagggcccatagacctccaacaaggtttgactagcctccagattgccatggatacagaccttagggcccttcaagactccataagtaaactagaggattccttaacctccctgtctgaagtagtgctccagaataggagaggccttgatctgctatttttgaaggaagggggcctttgtgcagccctaaaagaggaatgctgtttctatgttgaccactcaggcgcggtgcgagactccatgaggagactcaaggaaaggttagataagaggcagttagagcaccaaaagaatttaagttggtacgagggatggttcaaccgttccccctggcttactactttactgtctgcccttgctggtcccctgctactcctccttctgttactcaccctcgggccttgtgtcatcaataagttagtgcaattcatcaatgatagggttagtgcagtaaggattctgtaa

SEQ ID NO: 2 - protein, 644 aa, synthetic construct
  REGION 1..644: note = KoRV-A lacking C-terminal R-peptide
MLLISNPRHLGHPMSPGNWKRLIILLSCVFGGAEMNQQHNNPHQPMTLTWQVLSQTGSVVWEKKAVEPPWTWWPSLEPDVCALVAGLESWDIPELTASASQQARPPDSNYEHAYNQITWGTLGCSYPRARTRIARSQFYVCPRDGRSLSEARRCGGLESLYCKEWGCETAGTAYWQPRSSWDLITVGQGHPTGTCERTGWCNPLKIEFTEPGKRFRNWLQGRTWGLRFYVTGHPGVQLTIRLVITSPPPVVVGPDPVLAEQGPPRKIPFLPRVPVPTLSPPASPIPTVQASPPAPSTPSPTTGDRLFGLVQGAFLALNATNPEATESCWLCLALGPPYYEGIATPGQVTYASTDSQCRWGGKGKLTLTEVSGLGLCIGKVPPTHQHLCNLTIPLNASHTHKYLLPSNRSWWACNSGLTPCLSTSVFNQSNDFCIQIQLVPRIYYHPDGTLLQAYESPHSRNKREPVSLTLAVLLGLGVAAGIGTGSTALIKGPIDLQQGLTSLQIAMDTDLRALQDSISKLEDSLTSLSEVVLQNRRGLDLLFLKEGGLCAALKEECCFYVDHSGAVRDSMRRLKERLDKRQLEHQKNLSWYEGWFNRSPWLTTLLSALAGPLLLLLLLLTLGPCVINKLVQFINDRVSAVRIL

SEQ ID NO: 3 - DNA, 1956 bp, synthetic construct
  misc_feature 1..1956: note = KoRV-B lacking C-terminal R-peptide
  source 1..1956: mol_type = other DNA
atgcttctcatctcaaacccgcgccacctggggcacccgatgagtcctgggaactggaaaagactgatcatcctcttaagttgcgtgttcggcggcgccgaaatgaatcaacagcataacaaccctcaccaacccatgactctcacctggcaggtactgtcccagacgggaagtgtcgtttgggaaaagaaagcagtcgagccaccctggacgtggtggccctctcttgagcctgatgtgtgtgcgctggcggcgggtcttgagacctggggcatcccgcacctcaccgcgccagaatctcaacagtctgcgccccccgatggctgtggggtgctttatagtcaggtcggcagagggttggtcaggcaatcgagctatggaaccttggggtgtcgctgtccccgagaccggaacagactggctcaatctcagttctatgtgtgtccccgagatggccgttccctgtccgaggcctggaggtgcggagggtttgagtctttgtattgtaaagagtggggttgtgagaccacgggaaccgcctactggcaaccccggtcctcgtgggaccttataactgtaggcctgggccaccctacggggacatgcgaacacaccggttggtgcaacccactcaagatagagttcaccgagcctggcaaacggttccgaaactggctacaggggcgaacctggggtctaaggttctatgtgactggacacccaggcgtgcagttgaccatacgactagtgatcacaagtcccccaccggtagtagtgggccccgatcctgtcctcgcagaacaaggacctcccagaaaaattcccttcctccccagagtgccggttcctaccctatcacccccagcgtcccctatccccacggtacaggcaagccccccggcccctagtactccatctcccaccacgggcgaccggctctttggcctcgtacaaggggcctttctggccttgaatgccaccaacccggaggccacggagtcttgctggctctgtctagccctcggccctccttattacgaaggaatcgcaactccagggcaagtcacttatgcctccacagattcccaatgtcgctggggaggaaaagggaagctcaccctcactgaagtctctggactcgggttatgtatagggaaagtacctcctactcaccaacatctctgcaacctgactatacccttaaacgcctcacatactcacaagtacctcctcccctccaaccacagctggtgggcttgtaattctggcctcaccccctgcctctctacatcagtcttcaaccagtctaacgatttctgtatccagatccagcttgtccctcgcatctactatcacccagacggtaccttgctacaggcctatgagtccccccaccctagaaacaagagagagcctgtctcactcaccctggctgtccttctcggattaggggtcgcagcaggtataggtaccggctcgaccgccctaataaaagggcccatagacctccaacaaggtttgactagcctccaaattgccatggatacagaccttagagcccttcaagactccgtaagtaaactagagaactcattaacctccctttctgaagtagtgctccagaataggagaggccttgatctgctatttttgaaggaagggggcctttgtgcagccctaaaagaggagtgctgtttctatgttgaccactcaggcgcggtgcgagactccatgaggagactcaaggaaaggttagataagaggcagttagagcaccaaaagaatttaagttggtacgagggatggttcaaccgttccccctggcttactactttactgtctgcccttgctggtcccctgctactcctccttctgttactcaccctcgggccttgtgtcatcaataagttagtgcaattcatcaatgatagggttagtgcagtaaggattctgtaa

SEQ ID NO: 4 - protein, 651 aa, synthetic construct
  REGION 1..651: note = KoRV-B lacking C-terminal R-Peptide
MLLISNPRHLGHPMSPGNWKRLIILLSCVFGGAEMNQQHNNPHQPMTLTWQVLSQTGSVVWEKKAVEPPWTWWPSLEPDVCALAAGLETWGIPHLTAPESQQSAPPDGCGVLYSQVGRGLVRQSSYGTLGCRCPRDRNRLAQSQFYVCPRDGRSLSEAWRCGGFESLYCKEWGCETTGTAYWQPRSSWDLITVGLGHPTGTCEHTGWCNPLKIEFTEPGKRFRNWLQGRTWGLRFYVTGHPGVQLTIRLVITSPPPVVVGPDPVLAEQGPPRKIPFLPRVPVPTLSPPASPIPTVQASPPAPSTPSPTTGDRLFGLVQGAFLALNATNPEATESCWLCLALGPPYYEGIATPGQVTYASTDSQCRWGGKGKLTLTEVSGLGLCIGKVPPTHQHLCNLTIPLNASHTHKYLLPSNHSWWACNSGLTPCLSTSVFNQSNDFCIQIQLVPRIYYHPDGTLLQAYESPHPRNKREPVSLTLAVLLGLGVAAGIGTGSTALIKGPIDLQQGLTSLQIAMDTDLRALQDSVSKLENSLTSLSEVVLQNRRGLDLLFLKEGGLCAALKEECCFYVDHSGAVRDSMRRLKERLDKRQLEHQKNLSWYEGWFNRSPWLTTLLSALAGPLLLLLLLLTLGPCVINKLVQFINDRVSAVRIL

SEQ ID NO: 5 - DNA, 1980 bp, Koala retrovirus
  source 1..1980: mol_type = genomic DNA
atgcttctcatctcaaacccgcgccacctggggcacccgatgagtcctgggaactggaaaagactgatcatcctcttaagttgcgtgttcggcggcgccgaaatgaatcaacagcataacaaccctcaccaacccatgactctcacctggcaggtactgtcccagacgggaagtgtcgtttgggaaaagaaagcagtcgagccaccctggacgtggtggccctctcttgagcctgatgtgtgcgcgctggtagccggtcttgagtcctgggacatcccggaactcactgcatcggcctctcaacaggccaggccccctgattcaaactatgaacatgcttataatcagatcacttggggaaccctgggatgtagctatccccgggccagaacaagaattgctaggtcccagttctacgtgtgcccccgggatggccgttccctgtccgaggcccggaggtgcggaggacttgaatctttgtactgtaaagagtggggttgtgagaccgcgggaaccgcctactggcaaccccggtcctcgtgggaccttataactgtaggccagggtcaccctacagggacatgcgagcgcaccggttggtgcaacccactcaagatagagttcaccgagcctggcaaacggttccgaaactggctacaggggcgaacctggggtctaaggttctatgtgactggacacccaggcgtgcagttgaccatacgactagtgatcacaagtcccccaccggtagtagtgggccccgatcctgtcctcgcagaacaaggacctcccagaaaaattcccttcctccccagagtgccggttcctaccctatcacccccagcgtcccctatccccacggtacaggctagccccccggcccctagtactccatctcccaccacgggcgaccggctctttggcctcgtacaaggggcctttctggccttgaatgccaccaacccggaggccacggagtcttgctggctctgtctagccctcggccctccttattacgaaggaatcgcaactccagggcaagtcacttatgcctccacagattcccaatgtcgctggggaggaaaagggaagctcaccctcactgaagtctctggactcgggttatgtatagggaaagtacctcctactcaccaacatctctgcaacctgactatacccttaaacgcctcacatactcacaagtacctcctcccctccaaccgcagctggtgggcttgtaattctggcctcaccccctgcctctctacatcagtcttcaaccagtctaacgatttctgtatccagatccagcttgtccctcgcatctactatcacccagacggtaccttgctacaggcctatgagtccccccactctagaaacaagagagagcctgtctcactcaccctggctgtccttctcggattaggggtcgcagcaggtataggtaccggctcgaccgccctaataaaagggcccatagacctccaacaaggtttgactagcctccagattgccatggatacagaccttagggcccttcaagactccataagtaaactagaggattccttaacctccctgtctgaagtagtgctccagaataggagaggccttgatctgctatttttgaaggaagggggcctttgtgcagccctaaaagaggaatgctgtttctatgttgaccactcaggcgcggtgcgagactccatgaggagactcaaggaaaggttagataagaggcagttagagcaccaaaagaatttaagttggtacgagggatggttcaaccgttccccctggcttactactttactgtctgcccttgctggtcccctgctactcctccttctgttactcaccctcgggccttgtgtcatcaataagttagtgcaattcatcaatgatagggttagtgcagtaaggattctggttctcaggcacaagtaccagaccctagacaacgaggataacctttaa

SEQ ID NO: 6 - protein, 659 aa, Koala retrovirus
MLLISNPRHLGHPMSPGNWKRLIILLSCVFGGAEMNQQHNNPHQPMTLTWQVLSQTGSVVWEKKAVEPPWTWWPSLEPDVCALVAGLESWDIPELTASASQQARPPDSNYEHAYNQITWGTLGCSYPRARTRIARSQFYVCPRDGRSLSEARRCGGLESLYCKEWGCETAGTAYWQPRSSWDLITVGQGHPTGTCERTGWCNPLKIEFTEPGKRFRNWLQGRTWGLRFYVTGHPGVQLTIRLVITSPPPVVVGPDPVLAEQGPPRKIPFLPRVPVPTLSPPASPIPTVQASPPAPSTPSPTTGDRLFGLVQGAFLALNATNPEATESCWLCLALGPPYYEGIATPGQVTYASTDSQCRWGGKGKLTLTEVSGLGLCIGKVPPTHQHLCNLTIPLNASHTHKYLLPSNRSWWACNSGLTPCLSTSVFNQSNDFCIQIQLVPRIYYHPDGTLLQAYESPHSRNKREPVSLTLAVLLGLGVAAGIGTGSTALIKGPIDLQQGLTSLQIAMDTDLRALQDSISKLEDSLTSLSEVVLQNRRGLDLLFLKEGGLCAALKEECCFYVDHSGAVRDSMRRLKERLDKRQLEHQKNLSWYEGWFNRSPWLTTLLSALAGPLLLLLLLLTLGPCVINKLVQFINDRVSAVRILVLRHKYQTLDNEDNL

SEQ ID NO: 7 - DNA, 2001 bp, Koala retrovirus
  source 1..2001: mol_type = genomic DNA
atgcttctcatctcaaacccgcgccacctggggcacccgatgagtcctgggaactggaaaagactgatcatcctcttaagttgcgtgttcggcggcgccgaaatgaatcaacagcataacaaccctcaccaacccatgactctcacctggcaggtactgtcccagacgggaagtgtcgtttgggaaaagaaagcagtcgagccaccctggacgtggtggccctctcttgagcctgatgtgtgtgcgctggcggcgggtcttgagacctggggcatcccgcacctcaccgcgccagaatctcaacagtctgcgccccccgatggctgtggggtgctttatagtcaggtcggcagagggttggtcaggcaatcgagctatggaaccttggggtgtcgctgtccccgagaccggaacagactggctcaatctcagttctatgtgtgtccccgagatggccgttccctgtccgaggcctggaggtgcggagggtttgagtctttgtattgtaaagagtggggttgtgagaccacgggaaccgcctactggcaaccccggtcctcgtgggaccttataactgtaggcctgggccaccctacggggacatgcgaacacaccggttggtgcaacccactcaagatagagttcaccgagcctggcaaacggttccgaaactggctacaggggcgaacctggggtctaaggttctatgtgactggacacccaggcgtgcagttgaccatacgactagtgatcacaagtcccccaccggtagtagtgggccccgatcctgtcctcgcagaacaaggacctcccagaaaaattcccttcctccccagagtgccggttcctaccctatcacccccagcgtcccctatccccacggtacaggctagccccccggcccctagtactccatctcccaccacgggcgaccggctctttggcctcgtacaaggggcctttctggccttgaatgccaccaacccggaggccacggagtcttgctggctctgtctagccctcggccctccttattacgaaggaatcgcaactccagggcaagtcacttatgcctccacagattcccaatgtcgctggggaggaaaagggaagctcaccctcactgaagtctctggactcgggttatgtatagggaaagtacctcctactcaccaacatctctgcaacctgactatacccttaaacgcctcacatactcacaagtacctcctcccctccaaccacagctggtgggcttgtaattctggcctcaccccctgcctctctacatcagtcttcaaccagtctaacgatttctgtatccagatccagcttgtccctcgcatctactatcacccagacggtaccttgctacaggcctatgagtccccccaccctagaaacaagagagagcctgtctcactcaccctggctgtccttctcggattaggggtcgcagcaggtataggtaccggctcgaccgccctaataaaagggcccatagacctccaacaaggtttgactagcctccaaattgccatggatacagaccttagagcccttcaagactccgtaagtaaactagagaattccttaacctccctttctgaagtagtgctccagaataggagaggccttgatctgctatttttgaaggaagggggcctttgtgcagccctaaaagaggagtgctgtttctatgttgaccactcaggcgcggtgcgagactccatgaggagactcaaggaaaggttagataagaggcagttagagcaccaaaagaatttaagttggtacgagggatggttcaaccgttccccctggcttactactttactgtctgcccttgctggtcccctgctactcctccttctgttactcaccctcgggccttgtgtcatcaataagttagtgcaattcatcaatgatagggttagtgcagtaaggattctggttctcaggcacaagtaccagaccctagacaacgaggataacctttaa

SEQ ID NO: 8 - protein, 666 aa, Koala retrovirus
MLLISNPRHLGHPMSPGNWKRLIILLSCVFGGAEMNQQHNNPHQPMTLTWQVLSQTGSVVWEKKAVEPPWTWWPSLEPDVCALAAGLETWGIPHLTAPESQQSAPPDGCGVLYSQVGRGLVRQSSYGTLGCRCPRDRNRLAQSQFYVCPRDGRSLSEAWRCGGFESLYCKEWGCETTGTAYWQPRSSWDLITVGLGHPTGTCEHTGWCNPLKIEFTEPGKRFRNWLQGRTWGLRFYVTGHPGVQLTIRLVITSPPPVVVGPDPVLAEQGPPRKIPFLPRVPVPTLSPPASPIPTVQASPPAPSTPSPTTGDRLFGLVQGAFLALNATNPEATESCWLCLALGPPYYEGIATPGQVTYASTDSQCRWGGKGKLTLTEVSGLGLCIGKVPPTHQHLCNLTIPLNASHTHKYLLPSNHSWWACNSGLTPCLSTSVFNQSNDFCIQIQLVPRIYYHPDGTLLQAYESPHPRNKREPVSLTLAVLLGLGVAAGIGTGSTALIKGPIDLQQGLTSLQIAMDTDLRALQDSVSKLENSLTSLSEVVLQNRRGLDLLFLKEGGLCAALKEECCFYVDHSGAVRDSMRRLKERLDKRQLEHQKNLSWYEGWFNRSPWLTTLLSALAGPLLLLLLLLTLGPCVINKLVQFINDRVSAVRILVLRHKYQTLDNEDNL